(19) United States
(12) Patent Application Publication
(10) Pub. No.: US 2007/0293538 A1
(43) Pub. Date: Dec. 20, 2007

(54) Pharmaceutical Composition And Methods For Treating Neurodegenerative Disorders

(75) Inventor: Adrian Hobden, Salt Lake City, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: MYRIAD GENETICS, INCORPORATED, SALT LAKE CITY, UT (US)

(21) Appl. No.: 11/578,650

(22) PCT Filed: Apr. 12, 2005
(86) PCT No.: PCT/US05/12258

§ 371 Date: Aug. 16, 2007

Related U.S. Application Data
(60) Provisional application No. 60/562,126, filed on Apr. 13, 2004.

(51) Int. Cl. A61K 31/19; A61K 31/135; A61K 31/34; A61P 25/28; A61K 31/445; A61K 31/15

(52) U.S. Cl. 514/315; 514/470; 514/570; 514/640; 514/646; 514/657

(57) ABSTRACT

The invention provides compositions and methods for treating neurodegenerative disorders. The method of the invention involves administering to an individual in need of treatment a composition having an R-NSAID or a derivative thereof and a SSRI. The methods and compositions of the invention are useful for treating and preventing neurodegenerative disorders such as Alzheimer's disease, dementia, and mild cognitive impairment.

### CROSS-REFERENCE TO RELATED APPLICATIONS

This application claims priority to U.S. Provisional Application Ser. No. 60/562,126 filed on Apr. 13, 2004, the content of which is incorporated herein by reference in its entirety.

### FIELD OF THE INVENTION

The invention generally relates to compositions and methods for the prevention and treatment of neurodegenerative disorders, and particularly to a composition having an R-NSAID and a selective serotonin reuptake inhibitor and methods of use thereof in treating or preventing diseases and disorders such as Alzheimer's disease and mild cognitive impairment.

### BACKGROUND OF THE INVENTION

Dementia is a brain disorder that seriously affects a person's ability to carry out normal daily activities. Among older people, Alzheimer's disease (AD) is the most common form of dementia and involves parts of the brain that control thought, memory, and language. Despite intensive research throughout the world, the causes of AD are still unknown and there is no cure. AD most commonly begins after the age of 60, with the risk of acquiring the disease increasing with age. Younger people can also get AD, but it is much less common. It is estimated that 3 percent of men and women ages 65 to 74 have AD. Almost half of those ages 85 and older may have the disease. A recent study indicated that in newly diagnosed AD patients over 60 years old that the survival period was about half that as compared to the control population (Larson et al. Ann. Int. Med. 140:501-509 (2004)). AD is not a normal part of aging. Alzheimer's disease is a complex disease that can be caused by genetic and environmental factors.

In 1906, Dr. Alois Alzheimer noticed changes in the brain tissue of a woman who had died of an unusual mental illness. In her brain tissue, he found abnormal clumps (now known as amyloid plaques) and tangled bundles of fibers (now known as neurofibrillary tangles) which, today, are considered the pathological hallmarks of AD. Other brain changes in people with AD have been discovered. For example, with AD, there is a loss of nerve cells in areas of the brain that are vital to memory and other mental abilities. Scientists have found that there are lower levels of chemicals in the brain that carry complex messages back and forth between nerve cells. AD may disrupt normal thinking and memory by blocking these messages between nerve cells.

Plaques and tangles are found in the same brain regions that are affected by neuronal and synaptic loss. Neuronal and synaptic loss is universally recognized as the primary cause of decline in cognitive function in AD patients. The number of tangles is more highly correlated with cognitive decline than amyloid load in patients with AD (Albert PNAS 93:13547-13551 (1996)). The cellular, biochemical, and molecular events responsible for neuronal and synaptic loss in AD are not known. A number of studies have demonstrated that amyloid can be directly toxic to neurons resulting in behavioral impairment (see, e.g., Iversen et al. Biochem. J. 311:1-16 (1995); Weiss et al. J. Neurochem. 62:372-375 (1994); Lorenzo et al. Ann N Y Acad. Sci. 777:89-95 (1996); and Storey et al. Neuropathol. Appl. Neurobiol. 2:81-97 (1999)). The toxicity of amyloid or tangles is potentially aggravated by activation of the complement cascade (Rogers et al. PNAS 21:10016-10020 (1992); Rozemuller et al. Res. Immunol. 6:646-9 (1992); Rogers et al. Res Immunol. 6:624-30 (1992); and Webster et al. J. Neurochem. 69(1):388-98 (1997)).

Evidence that amyloid β protein (Aβ) deposition causes some forms of AD was provided by genetic and molecular studies of some familial forms of AD (FAD). (See, e.g., Ii Drugs Aging 7(2):97-109 (1995); Hardy PNAS 94(6):2095-7 (1997); and Selkoe J. Biol. Chem. 271(31):18295-8 (1996)). The amyloid plaque buildup in AD patients suggests that abnormal processing of Aβ may be a cause of AD. Aβ is a peptide of 39 to 42 amino acids and is the core of senile plaques observed in all Alzheimer's disease cases. If abnormal processing is the primary cause of AD, then familial Alzheimer's disease (FAD) mutations that are linked (genetically) to FAD may induce changes that, in one way or another, foster Aβ deposition. Mutations in the FAD genes can result in increased Aβ deposition. It is noted that the vast majority of Alzheimer's disease cases are not a result of mutations in FAD genes.

The first of the FAD genes codes for the Aβ precursor, amyloid precursor protein (APP) (Selkoe J. Biol. Chem. 271(31):18295-8 (1996)). Mutations in the APP gene are very rare, but all of them cause AD with 100% penetrance and result in elevated production of either total Aβ or Aβ₄₂, both in model transfected cells and transgenic animals. Two other FAD genes code for presenilin 1 and 2 (PS1, PS2) (Hardy PNAS 94(6):2095-7 (1997)). The presenilins contain 8 transmembrane domains and several lines of evidence suggest that they are involved in intracellular protein trafficking. Other studies suggest that the presenilins function as proteases. Mutations in the presenilin genes are more common than in the APP genes, and all of them also cause FAD with 100% penetrance. Similar to APP mutants, studies have demonstrated that PS1 and PS2 mutations shift APP metabolism, resulting in elevated Aβ₄₂ production (in vitro and in vivo).

In the United States alone, four million adults suffer from Alzheimer's disease (AD). Not only is Alzheimer's disease significantly impacting the lives of countless families today, it is threatening to become even more of a problem as the baby boom generation matures. The economic burden of AD is estimated to cost over $100 billion a year and the average lifetime cost per patient is estimated to be $174,000. Unfortunately, there is no cure available for AD. Of the five drugs currently being used in the US for the treatment of AD, four of them—tacrine (Cognex®), donepezil (Aricept(®), rivastigmine (Exelon®), and galantamine (Reminyl®), are inhibitors of acetylcholine esterase. Another drug, memantine, was recently approved for treating moderate-to-severe AD. More recently it was reported that memantine showed efficacy in treating mild-to-moderate AD. Memantine is a NMDA receptor antagonist.

The drugs currently used for treating AD, including memantine and the acetylcholine esterase inhibitors, are marginally efficacious and have undesirable side-effects. Thus, there is a large unmet need for better and safer drugs.

### SUMMARY OF THE INVENTION

The invention generally relates to compositions and therapeutic treatments for neurodegenerative disorders. More specifically, the invention provides a composition for treating and delaying the onset of neurodegenerative disorders. The composition of the invention has one or more selective serotonin reuptake inhibitors (“SSRIs”) and one or more R-NSAIDs and optionally one or more pharmaceutically acceptable carriers. The method of the invention involves treating an individual in need of treatment, a therapeutically effective amount of one or more SSRIs and one or more R-NSAIDs.

In a first embodiment, the invention provides a composition comprising one or more SSRIs (including pharmaceutically acceptable salt and esters thereof) and one or more R-NSAIDs (including pharmaceutically acceptable salt and esters thereof). In one aspect of this embodiment, the SSRI is sertraline. In another aspect of this embodiment, the SSRI is selected from the group consisting of fluoxetine, fluvoxamine, paroxetine, sertraline, citalopram, and escitalopram, and pharmaceutically acceptable salts thereof. In one aspect of this embodiment the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etolodac, and pharmaceutically acceptable salt and esters thereof. In yet another aspect of this embodiment, the SSRI is selected from the group consisting of fluoxetine, fluvoxamine, paroxetine, sertraline, citalopram, and escitalopram, and pharmaceutically acceptable salt and esters thereof, and the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etodolac, and pharmaceutically acceptable salt and esters thereof. In still another aspect of this embodiment, the R-NSAID is R-flurbiprofen or a pharmaceutically acceptable salt or ester thereof. In another aspect, the R-NSAID is R-flurbiprofen or a pharmaceutically acceptable salt or ester thereof and the SSRI is selected from the group consisting of fluoxetine, fluvoxamine, paroxetine, sertraline, citalopram, and escitalopram, or a pharmaceutically acceptable salt or ester thereof. The invention further provides compositions having R-flurbiprofen or a pharmaceutically acceptable salt or ester thereof and fluoxetine or a pharmaceutically acceptable salt thereof; R-flurbiprofen or a pharmaceutically acceptable salt or ester thereof and fluvoxamine or a pharmaceutically acceptable salt thereof; R-flurbiprofen or a pharmaceutically acceptable salt or ester thereof and paroxetine or a pharmaceutically acceptable salt thereof; R-flurbiprofen or a pharmaceutically acceptable salt or ester thereof and sertraline or a pharmaceutically acceptable salt thereof; R-flurbiprofen or a pharmaceutically acceptable salt or ester thereof and citalopram or a pharmaceutically acceptable salt thereof; R-flurbiprofen or a pharmaceutically acceptable salt or ester thereof and escitalopram or a pharmaceutically acceptable salt thereof. The compositions of this embodiment can be provided with the two active ingredients combined together in a single dose with one or more pharmaceutically acceptable carriers, or as two separate pharmaceutical compositions contained in appropriate packaging indicating the two pharmaceuticals are to be used together.

In a second embodiment, the invention provides a method for treating neurodegenerative disorders. According to the method of this embodiment, a therapeutically effective amount of one or more R-NSAIDs (including pharmaceutically acceptable salts and esters thereof) and one or more SSRIs (including pharmaceutically acceptable salts thereof) is administered to an individual in need of such treatment. The individual in need of treatment can have a neurodegenerative disorder, a predisposition to a neurodegenerative disorder, and/or desire prophylaxis against or a delay in the onset of, neurodegenerative disorders. In one aspect of this embodiment, the effective amount of the one or more R-NSAIDs and one or more SSRIs is capable of reducing at least one symptom of the neurodegenerative disorder. In another aspect, for individuals desiring prophylaxis against a neurodegenerative disorder, the effective amount of the one or more R-NSAIDs and one or more SSRIs, is capable of preventing an increase (or slowing the rate of increase) in or delaying the onset of, at least one symptom of the neurodegenerative disorder. In one aspect of this method, the SSRI is sertraline or a pharmaceutically acceptable salt thereof. In another aspect of this method, the one or more SSRIs are selected from the group consisting of fluoxetine, fluvoxamine, paroxetine, sertraline, citalopram, and escitalopram, and pharmaceutically acceptable salts thereof. In one aspect of this method, the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etodolac, and pharmaceutically acceptable salts and esters thereof. In yet another aspect of this method, the SSRI is selected from the group consisting of fluoxetine, fluvoxamine, paroxetine, sertraline, citalopram, and escitalopram, and pharmaceutically acceptable salts thereof, and the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etodolac, and pharmaceutically acceptable salts and esters thereof. In still another aspect of this method, the R-NSAID is R-flurbiprofen or a pharmaceutically acceptable salt thereof. In another aspect of this method the R-NSAID is R-flurbiprofen or a pharmaceutically acceptable salt thereof and the SSRI is selected from the group consisting of fluoxetine, fluvoxamine, paroxetine, sertraline, citalopram, and escitalopram and pharmaceutically acceptable salts thereof. The method of the invention further provides for the treatment or prophylaxis or delay the onset of neurodegenerative disorders by administering an effective amount of R-flurbiprofen or a pharmaceutically acceptable salt or ester thereof and a SSRI; R-flurbiprofen or a pharmaceutically acceptable salt or ester thereof and fluoxetine or a pharmaceutically acceptable salt thereof; R-flurbiprofen or a pharmaceutically acceptable salt or ester thereof and fluvoxamine or a pharmaceutically acceptable salt thereof; R-flurbiprofen or a pharmaceutically acceptable salt or ester thereof and paroxetine or a pharmaceutically acceptable salt thereof; R-flurbiprofen or a pharmaceutically acceptable salt or ester thereof and sertraline or a pharmaceutically acceptable salt thereof; R-flurbiprofen or a pharmaceutically acceptable salt or ester thereof and citopram or a pharmaceutically acceptable salt thereof; R-flurbiprofen or a pharmaceutically acceptable salt or ester thereof and escitalopram or a pharmaceutically acceptable salt or ester thereof. In a preferred aspect of this method, the neurodegenerative disease is selected from the group consisting of Alzheimer's disease, dementia, mild cognitive impairment, and tauopathies (e.g., corticobasal degeneration, frontotemporal dementia with Parkinsonism linked to chromosome 17, and progressive supranuclear palsy). In another preferred embodiment, the invention provides a method for the treatment or prophylaxis or delay the onset of Alzheimer's disease or MCI through the administration of an effective amount of R-flurbiprofen or a pharmaceutically acceptable salt or ester thereof and fluoxetine or a pharmaceutically acceptable salt thereof; R-flurbiprofen or a pharmaceutically acceptable salt or ester thereof and fluvoxamine or a pharmaceutically acceptable salt thereof; R-flurbiprofen or a pharmaceutically acceptable salt or ester thereof and paroxetine or a pharmaceutically acceptable salt thereof; R-flurbiprofen or a pharmaceutically acceptable salt or ester thereof and sertraline or a pharmaceutically acceptable salt thereof; R-flurbiprofen or a pharmaceutically acceptable salt or ester thereof and citalopram or a pharmaceutically acceptable salt thereof; R-flurbiprofen or a pharmaceutically acceptable salt or ester thereof and escitalopram or a pharmaceutically acceptable salt thereof. It is preferred that the combination therapy provides a lessening in decline in cognitive function. Preferably, the lessening in decline in cognitive function is at least 25% as compared to individuals treated with placebo, more preferably at least 40%, and even more desirably at least 60%. For example, an individual treated with placebo having probable mild-to-moderate Alzheimer's disease is expected to score approximately 5.5 points worse on the ADAS-cog test after a specified period of time of treatment (e.g., 1 year) whereas an individual treated with the composition of this aspect of the invention for the same period of time will score approximately 2.2 points worse on the ADAS-cog scale with a 60% decrease in decline or 3.3 points worse with a 40% decrease in decline in cognitive function when treated with the composition for the same specified period of time.

In a third embodiment, the invention provides a method of reducing amyloid β₄₂ (Aβ₄₂) protein levels in a mammal, for example in brain or in a body fluid such as CSF or plasma or blood. In particular, the method relates to reducing, lowering, or slowing or preventing an increase in Aβ₄₂ protein levels, in an individual in need of such treatment, by administering to the individual a therapeutically effective amount of one or more R-NSAIDs (including pharmaceutically acceptable salts and esters thereof) and one or more SSRIs (including pharmaceutically acceptable salts and esters thereof). The individual in need of treatment can have a neurodegenerative disorder, a predisposition to a neurodegenerative disorder, and/or a desire for delay the onset of or prophylaxis against neurodegenerative disorders, where the disorder is characterized by increased Aβ₄₂ protein levels. In one aspect, the effective amount is an amount of one or more R-NSAIDs and one or more SSRIs that is sufficient for reducing Aβ₄₂ protein levels (or is sufficient for slowing the rate of increase). In a preferred aspect, the effective amount is an amount of one or more R-NSAIDs and one or more SSRIs that is sufficient for reducing Aβ₄₂ protein levels. In another aspect, for individuals desiring prophylaxis against a neurodegenerative disorder, the effective amount is an amount of one or more R-NSAIDs and one or more SSRIs that is sufficient for preventing an increase in Aβ₄₂ protein levels or an increase in the rate of Aβ₂ increase. In one aspect of this method, the SSRI is sertraline. In another aspect of this method, the SSRI is selected from the group consisting of fluoxetine, fluvoxamine, paroxetine, sertraline, citalopram, and escitalopram oxalate, and pharmaceutically acceptable salts thereof. In one aspect of this method, the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etodolac, and pharmaceutically acceptable salts and esters thereof. In yet another aspect of this method, the SSRI is selected from the group consisting of fluoxetine, fluvoxamine, paroxetine, sertraline, citalopram, and escitalopram and pharmaceutically acceptable salts thereof and the R-NSAID is selected from the group consisting of R-flurbiprofen, R-ketoprofen, R-ketorolac, R-naproxen, R-tiaprofenic acid, R-suprofen, R-carprofen, R-pirprofen, R-indoprofen, R-benoxaprofen, and R-etodolac and pharmaceutically acceptable salts and esters thereof. In still another aspect of this method, the R-NSAID is R-flurbiprofen or a pharmaceutically acceptable salt or ester thereof. In another aspect of this method, the R-NSAID is R-flurbiprofen or a pharmaceutically acceptable salt or ester thereof and the S SRI is selected from the group consisting of fluoxetine, fluvoxamine, paroxetine, sertraline, citalopram, and escitalopram oxalate and pharmaceutically acceptable salts thereof. The method of the invention further provides for the treatment or prophylaxis or delaying the onset of neurodegenerative disorders with an Aβ₄₂ protein lowering effective amount of R-flurbiprofen or a pharmaceutically acceptable salt or ester thereof and fluoxetine or a pharmaceutically acceptable salt thereof; R-flurbiprofen or a pharmaceutically acceptable salt or ester thereof and fluvoxamine or a pharmaceutically acceptable salt thereof; R-flurbiprofen or a pharmaceutically acceptable salt or ester thereof and paroxetine or a pharmaceutically acceptable salt thereof; R-flurbiprofen or a pharmaceutically acceptable salt or ester thereof and sertraline or a pharmaceutically acceptable salt thereof; R-flurbiprofen or a pharmaceutically acceptable salt or ester thereof and citalopram or a pharmaceutically acceptable salt thereof; R-flurbiprofen or a pharmaceutically acceptable salt or ester thereof and escitalopram or a pharmaceutically acceptable salt thereof. In a preferred aspect of this method, the neurodegenerative disease is selected from the group consisting of Alzheimer's disease, dementia, mild cognitive impairment, and tauopathies (e.g., corticobasal degeneration, frontotemporal dementia with Parkinsonism linked to chromosome 17, and progressive supranuclear palsy). In another preferred embodiment, the invention provides a method for the treatment or prophylaxis or delaying the onset of Alzheimer's disease or MCI through administration, to an individual in need of treatment, of an Aβ₄₂ protein lowering effective amount of R-flurbiprofen and fluoxetine; R-flurbiprofen and fluvoxamine; R-flurbiprofen and paroxetine; R-flurbiprofen and sertraline; R-flurbiprofen and citalopram; R-flurbiprofen and escitalopram oxalate.

In fourth embodiment, the invention provides compositions and methods for treating and/or preventing or delaying the onset of neurodegenerative disorders by administering, to an individual in need of such treatment, an effective amount of one or more R-NSAIDs (including pharmaceutically acceptable salts or esters), one or more SSRIs such as fluoxetine, fluvoxamine, paroxetine, sertraline, citalopram, and escitalopram oxalate (or pharmaceutically acceptable salts or esters), and one or more compounds selected from the group consisting of secretase inhibitors, acetylcholine esterase inhibitors, GABA-A alpha 5 inverse agonists, NMDA antagonists (i.e., memantine) and antioxidants. The combination can be administered simultaneously or separately.

In a fifth embodiment, the invention provides a method of lowering Aβ₄₂ levels to a greater extent than inhibiting COX-1, COX-2, or a combination thereof. In particular, the method of this embodiment involves administering to a patient, in need of treatment, an effective amount of one or more R-NSAIDs (or pharmaceutically acceptable salts or esters thereof) and one or more SSRIs (or pharmaceutically acceptable salts or esters thereof). According to one aspect of this embodiment, the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etodolac, and pharmaceutically acceptable salts or esters thereof. According to another aspect of this embodiment, the SSRI is selected from the group consisting of fluoxetine, fluvoxamine, paroxetine, sertraline, citalopram, and escitalopram oxalate pharmaceutically acceptable salts thereof. In another aspect of this embodiment, the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etodolac, and pharmaceutically acceptable salts or esters thereof, and the SSRI is selected from the group consisting of fluoxetine, fluvoxamine, paroxetine, sertraline, citalopram, and escitalopram oxalate, pharmaceutically acceptable salts thereof. In a preferred aspect of this embodiment, the R-NSAID is R-flurbiprofen or a pharmaceutically acceptable salt or ester thereof and the SSRI is selected from the group consisting of fluoxetine, fluvoxamine, paroxetine, sertraline, citalopram, and escitalopram oxalate and pharmaceutically acceptable salts thereof. The method of this embodiment involves the lowering of Aβ42 levels while not substantial affecting the activity of COX-1, COX-2, or both COX-1 and COX-2. Thus, the amount that is administered is effective for lowering Aβ42 levels and does not substantially inhibit COX-1, COX-2, or both COX-1 and COX-2. For example, the effective amount can be above the ED₅₀ (the dose therapeutically effective in 50% of the population) for Aβ₄₂ lowering, and below the ED₅₀ for COX inhibition. Another example is a sufficiently small amount of compound so that inhibition of at least one COX activity is negligible and Aβ₄₂ levels are reduced. The method of this embodiment can be used to treat and/or prevent Alzheimer's disease. The method of this embodiment can also be used to treat and/or prevent MCI, dementia, and other neurodegenerative disorders.

In another embodiment, the invention provides a therapeutic method which comprises identifying a patient diagnosed of depression and treating the patient with an effective amount of one or more R-NSAIDs (including pharmaceutically acceptable salts and esters thereof) and one or more SSRIs (including pharmaceutically acceptable salts thereof). In yet another embodiment, the invention provides a therapeutic method which comprises identifying a patient diagnosed of depression and a neurodegenerative disorder (such as Alzheimer's disease and MCI), and treating the patient with an effective amount of one or more R-NSAIDs (including pharmaceutically acceptable salts and esters thereof) and one or more SSRIs (including pharmaceutically acceptable salts thereof). In a further embodiment, the invention provides a therapeutic method which comprises identifying a patient diagnosed of depression and having a predisposition to a neurodegenerative disorder (such as Alzheimer's disease and MCI), and treating the patient with an effective amount of one or more R-NSAIDs (including pharmaceutically acceptable salts and esters thereof) and one or more SSRIs (including pharmaceutically acceptable salts thereof). In such embodiments, the effective amount of the one or more R-NSAIDs is an amount effective in treating or delaying the onset of Alzheimer's disease or in reducing Aβ-42 level in CSF or plasma, and the effective amount of one or more SSRIs is an amount effective in treating depression. Thus, the one or more SSRIs are selected from the group consisting of fluoxetine, fluvoxamine, paroxetine, sertraline, citalopram, and escitalopram, and pharmaceutically acceptable salts thereof. The R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etodolac, and pharmaceutically acceptable salts and esters thereof. Preferably, the R-NSAID is R-flurbiprofen or a pharmaceutically acceptable salt thereof.

In various embodiments and aspect of the present invention, the compositions are preferably substantially free of S-NSAID. In particular, a composition having R-flurbiprofen and an SSRI is substantially free of S-flurbiprofen or a salt or ester thereof.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### DETAILED DESCRIPTION OF THE INVENTION

The invention provides compositions and therapeutic treatments for neurodegenerative disorders, e.g., Alzheimer's disease, MCI, Down's syndrome, and tauopathies (e.g., corticobasal degeneration, frontotemporal dementia with Parkinsonism linked chromosome 17, and progressive supranuclear palsy, etc.). Specifically, the invention provides a composition having one or more SSRIs (including pharmaceutically acceptable salts and esters thereof) and one or more R-NSAIDs (including pharmaceutically acceptable salts and esters thereof). The invention provides a method that involves treating an individual in need of treatment with an effective amount of one or more SSRIs and one or more R-NSAIDs. The method of the invention can involve co-administering the one or more SSRIs and the one or more R-NSAIDs, or the one or more SSRIs and the one or more R-NSAIDs can be administered to the same individual at different times and/or by different routes of administration. For example, the SSRI can be administered in the morning and the R-NSAID can be administered in the evening. Advantageously, the combination of R-NSAID and SSRI can be administered together as described herein. Without wishing to be bound by any theory, it is believed that the R-NSAID-SSRI combination therapy has unexpected properties particularly useful for the treatment and prophylaxis of neurodegenerative disease like dementia, mild cognitive impairment, and/or Alzheimer's disease.

The compositions of the invention has one or more SSRIs (including pharmaceutically acceptable salts and esters thereof) and one or more R-NSAIDs (including pharmaceutically acceptable salts and esters thereof). The SSRI used in the invention can be sertraline. Preferred SSRIs are selected from the group consisting of fluoxetine, fluvoxamine, paroxetine, sertraline, citalopram, and escitalopram, and pharmaceutically acceptable salts thereof. Preferred R-NSAIDs are selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etodolac, and pharmaceutically acceptable salts and esters thereof. It is contemplated that nitrosylated and nitrosated NSAIDs (and R-NSAIDs) can also be used in the methods of the invention (see, e.g., U.S. Pat. Nos. 6,593,347; 5,703,073; and PCT application WO 94/12463 which are herein incorporated by reference in their entirety). Preferably, the SSRI is selected from the group consisting of fluoxetine, fluvoxamine, paroxetine, sertraline, citalopram, and escitalopram oxalate, and the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etodolac. A preferred composition of the invention has R-flurbiprofen and one or more SSRIs. Another preferred composition has R-flurbiprofen and one or more SSRIs selected from the group consisting of fluoxetine, fluvoxamine, paroxetine, sertraline, citalopram, and escitalopram oxalate. The invention further provides composition having R-flurbiprofen and fluoxetine; R-flurbiprofen and fluvoxamne; R-flurbiprofen and paroxetine; R-flurbiprofen and sertraline; R-flurbiprofen and citalopram; R-flurbiprofen and escitalopram oxalate. The compositions of this embodiment can be provided with the two active ingredients combined together in a single dose with one or more pharmaceutically acceptable carriers or as two separate pharmaceutical compositions contained in appropriate packaging indicating the two pharmaceuticals are to be used together. Without wishing to be bound by any theory, it is believed the compositions of the invention are unexpectedly useful for treating neurodegenerative disorders and can exhibit synergistic and/or unexpected effects when used in combination for treating neurodegenerative disorders or depression.

The pathological hallmarks of Alzheimer's disease are most prevalent in the brain regions involved in higher cognitive functions. These features include a marked loss of neurons and synapses, numerous extracellular neuritic (senile) plaques and intracellular neurofibrillary tangles. The plaques are formed by a core of amyloid material surrounded by a halo of dystrophic neurites. The major component of the core is a peptide of 37 to 43 amino acids in length called the amyloid beta protein (Aβ), the major forms being Aβ₄₀ and Aβ₄₂. The tangles are formed by paired helical filaments, the major component of which is a hyperphosphorylated form of the microtubule-associated protein tau (τ). A large body of evidence suggests that the metabolism of APP and the generation of the Aβ peptide are central in AD pathogenesis. In fact, APP metabolism is regarded as the biochemical link between the pathology and genetics of AD.

Accordingly, in a preferred embodiment, the invention provides methods for lowering or preventing an increase in Aβ₄₂ levels in an individual in need of such treatment. It is believed that by lowering the amounts of Aβ₄₂ in an individual by administering an Aβ₄₂ lowering effective amount of one or more R-NSAIDs (including derivatives thereof and pharmaceutically acceptable salts and esters thereof) and one or more SSRIs (including pharmaceutically acceptable salts and esters thereof), as described herein, that Alzheimer's disease, dementia, and mild cognitive impairment can be treated or prevented. Generally, the method relates to the idea that administering, to an individual, an effective amount of one or more R-NSAIDs and one or more SSRIs can lower Aβ₄₂ levels. Thus, diseases characterized by increased levels of Aβ₄₂, can be treated or prevented with the methods of this embodiment which are designed to lower Aβ₄₂ or prevent an increase in Aβ₄₂.

While not wishing to be bound by any theory, it is contemplated that administration of one or more R-NSAIDs, e.g., R-flurbiprofen, and one or more SSRIs, e.g., fluoxetine, fluvoxamine, paroxetine, sertraline, citalopram, or escitalopram oxalate, can act in vivo, synergistically to treat and/or prevent Alzheimer's disease, dementia, MCI by lowering the amount of Aβ₄₂ that is present or would be present in the absence of such treatment. Amyloid β polypeptides are derived from amyloid precursor proteins (APPs). A variety of amyloid P polypeptides are known including Aβ₃₄, Aβ₃₇, Aβ₃₈, Aβ₃₉, and Aβ₄₀. Increased Aβ₄₂ levels are associated with Alzheimer's disease, dementia, MCI. Thus, by lowering the amounts of Aβ₄₂, a treatment is provided for combating Alzheimer's disease and/or MCI. It is contemplated that the combination of R-flurbiprofen and SSRI can synergistically lessen the progression of symptoms of AD (or the rate of increase in the symptoms).

In addition, while not wishing to be bound by any theory, it is believed that the combination of R-NSAID and SSRI is capable of slowing the rate of death of neurons. Accordingly, it is also believed that the combination of R-NSAID and SSRI acts in vivo to treat and/or prevent Alzheimer's disease, MCI and depression by slowing the rate of death of neurons that is present or would be present in the absence of such treatment.

According to a preferred embodiment, the invention provides a method of lowering Aβ₄₂ levels to a greater extent than inhibiting COX-1, COX-2, or a combination thereof. In particular, the method of this embodiment comprises administering, to a patient in need of treatment, an effective amount of an R-NSAID (including pharmaceutically acceptable salts and esters thereof), e.g., R-flurbiprofen, and one or more SSRIs (including pharmaceutically acceptable salts and esters thereof) such as fluoxetine, fluvoxamine, paroxetine, sertraline, citalopram, and escitalopram oxalate, wherein the effective amount of composition is capable of lowering Aβ₄₂, while not substantially affecting or inhibiting the activity of at least one isoform of COX. Thus, the method of this embodiment involves the lowering of Aβ₄₂ levels while not substantially inhibiting the activity of COX-1, COX-2, or both COX-1 and COX-2. The method of this embodiment can be used to treat and/or prevent Alzheimer's disease, MCI, dementia, and/or other neurodegenerative disorders. In one aspect of this embodiment, the effective amount of the one or more R-NSAIDs, e.g., R-flurbiprofen and one or more SSRIs, such as fluoxetine, fluvoxamine, paroxetine, sertraline, citalopram, or escitalopram oxalate, reduces Aβ₄₂ levels or production of Aβ₄₂ by at least 1, 2, 5, 10, 15, 20, 25, 30, 40, or 50 or more percent while inhibiting COX-1, COX-2, or both COX-1 and COX-2 by less than 1, 2, 5, 10, 15, 20, 25, 30, 40, 50, 60, 70, 80, or 90 percent. In a preferred aspect of this embodiment, the effective amount of the R-NSAID, e.g., R-flurbiprofen, and one or more SSRIs, such as fluoxetine, fluvoxamine, paroxetine, sertraline, citalopram, or escitalopram oxalate, lowers Aβ₄₂ by at least 5 percent while not substantially inhibiting COX-1 , COX-2, or both COX-1 and COX-2 activity or levels. In another preferred aspect of this embodiment, the effective amount of the R-NSAID, e.g., R-flurbiprofen, and one or more SSRIs such as fluoxetine, fluvoxamine, paroxetine, sertraline, citalopram, or escitalopram oxalate, that is administered to an individual is such that it lowers Aβ₄₂ levels, and does not inhibit COX activity to a significant extent, e.g., the amount administered is below the in vivo IC₅₀ value for COX-1, COX-2 or both COX-1 and COX-2 and above the in vivo IC₅₀ value for Aβ₄₂ lowering activity. As used in this context, IC₅₀ refers to the concentration of compound or composition sufficient to inhibit COX activity by 50% (COX-1, COX-2, or both COX-1 and COX-2) or reduce Aβ₄₂ levels (or rates of production) by 50%. An “effective amount” according to this preferred aspect of this embodiment, can also be viewed in terms of ED₅₀ parameters, binding constants, dissociation constants, and other pharmacological parameters, e.g., the amount administered is below the ED₅₀ value for COX-1, COX-2 or both COX-1 and COX-2 and above the ED₅₀ value for Aβ₄₂. It is noted that the effective amount of the compound does not necessarily have to be above an IC₅₀ or ED₅₀ for Aβ₂ lowering and below the IC₅₀ or ED₅₀ for COX inhibition. That is, the “effective amount” can be at some intermediate value such that Aβ₄₂ levels (or rates of production) are lowered to a greater extent than inhibition of COX-1, COX-2 or both COX-1 and COX-2. In one aspect, the method of this embodiment is thought to avoid the liability of adverse side effects associated with COX-1 and COX-2 inhibitors.

In another embodiment, the combination therapy of the invention provides a lessening in decline in cognitive function that is at least 25% as compared to individuals treated with placebo, more preferably at least 40%, and even more desirably at least 60%. For example, an individual treated with placebo having probable mild-to-moderate Alzheimer's disease is expected to score approximately 5.5 points worse on the ADAS-cog test after a specified period of time of treatment (e.g., 1 year) whereas an individual treated with the composition of this aspect of the invention for the same period of time will score approximately 2.2 points worse on the ADAS-cog scale with a 60% decrease in decline or 3.3 points worse with a 40% decrease in decline in cognitive function when treated with the composition for the same specified period of time.

In another embodiment, the invention provides a method of lowering Aβ₄₂ levels and increasing Aβ₃₈ levels, while not affecting Aβ₄₀ levels. The method of this embodiment comprises administering, to an individual in need of such treatment, an effective amount of an R-NSAID (including pharmaceutically acceptable salts and esters thereof), e.g., R-flurbiprofen, and one or more SSRIs (including pharmaceutically acceptable salts and esters thereof) such as fluoxetine, fluvoxamine, paroxetine, sertraline, citalopram, or escitalopram oxalate. The method according to this embodiment is useful for preventing and treating Alzheimer's disease. It is also contemplated that the method of this embodiment is useful for treating and preventing other disorders such as MCI, dementia, other neurodegenerative disorders. The Aβ₄₂ lowering method of this embodiment can also increase the levels of other Aβ proteins smaller than Aβ_{40,} including Aβ_{34,} Aβ_{37,} Aβ_{38,} and Aβ₃₉.

In another embodiment, the invention relates to a method of preventing or delaying the onset of Alzheimer's disease or MCI. According to this embodiment, a method is provided which comprises treating an individual in need of such treatment, with a prophylactically effective amount of an R-NSAID (including pharmaceutically acceptable salts and esters thereof), e.g., R-flurbiprofen, and one or more SSRIs (including pharmaceutically acceptable salts and esters thereof) such as fluoxetine, fluvoxamine, paroxetine, sertraline, citalopram, or escitalopram oxalate. The method of this embodiment is useful for preventing or delaying the symptoms of Alzheimer's disease, the onset of Alzheimer's disease, and/or the progression of the disease.

The invention provides, in yet another embodiment, a method of decreasing cognitive decline in a patient in need of such treatment. The method of this embodiment involves treating an individual desiring (or needing) a slowing or decrease in decline in cognitive function, with an effective amount of an R-NSAID (including pharmaceutically acceptable salts and esters thereof), i.e., R-flurbiprofen, and a SSRI (including pharmaceutically acceptable salts and esters thereof). Preferably the SSRI is fluoxetine, fluvoxamine, paroxetine, sertraline, citalopram, or escitalopram oxalate.

In one embodiment, a patient suspected of having mild-to-moderate Alzheimer's disease is identified using diagnostic techniques readily available to the skilled practitioner (i.e., MMSE score of >15and <26, has a diagnosis of dementia according to DSM IV (TR) and/or meets the NINCDS-ADRDA criteria for probable AD). The patient is then administered, on a daily basis, or twice daily basis, an Alzheimer's disease treating therapeutically effective amount of R-flurbiprofen and a SSRI. Preferably the SSRI is fluoxetine, fluvoxamine, paroxetine, sertraline, citalopram, or escitalopram oxalate.

It is noted that the combination treatment can be applied to a patient for purposes of treating any suitable diseases and disorders, including but not limited to, dementia, Alzheimer's disease, mild cognitive impairment (MCI), tauopathies (e.g., corticobasal degeneration, frontotemporal dementia with Parkinsonism linked to chromosome 17, and progressive supranuclear palsy), Down's Syndrome, rheumatoid arthritis, inflammatory disorders, depression, Parkinson's disease, manic disorders and others. Thus, the patient treated can have one of the diseases and disorders requiring treatment, or have two or more of the diseases and disorders. For example, the compositions and combination treatment of the present invention can be particularly desirable if a patient desires treatment (therapeutic treatment and/or delaying the onset) of a first disease selected from dementia, Alzheimer's disease, mild cognitive impairment (MCI), tauopathies (e.g., corticobasal degeneration, frontotemporal dementia with Parkinsonism linked to chromosome 17, and progressive supranuclear palsy), and Down's Syndrome, and a second disease selected from depression and manic disorder. In particular, the compositions and combination treatment of the present invention can be advantageous in the treatment of a patient having, and/or desiring a delay in the onset of, both Alzheimer's disease or MCI, and depression.

Preferably, the daily dosage of R-flurbiprofen is from about 5 mg to about 2000 mg, more preferably from about 50 mg to about 1800 mg and even more preferably from about 200 to about 1600 mg. Preferably, the daily dosage of the SSRI is as follows: from about 1 mg to 250 mg fluoxetine; from about 1 mg to about 500 mg of fluvoxamine; from about 1 mg to 200 mg of paroxetine; from about 1 mg to about 500 mg of sertraline; from about 1 mg to about 200 mg citalopram; from about 1 mg to about 50 mg escitalopram oxalate. More preferably, the daily dosage of SSRI is as follows: from about 20 mg to 80 mg fluoxetine; from about 50 mg to about 300 mg of fluvoxamine; from about 20 mg to 60 mg of paroxetine; from about 50 mg to about 200 mg of sertraline; from about 20 mg to about 60 mg citalopram; from about 10 mg to about 20 mg escitalopram oxalate. Even more preferably, the daily dosage of the SSRI is as follows: from about 30 mg to 50 mg fluoxetine; from about 100 mg to about 200 mg of fluvoxamine; from about 30 mg to 50 mg of paroxetine; from about 100 mg to about 150 mg of sertraline; from about 30 mg to about 50 mg citalopram; from about 12 mg to about 18 mg escitalopram oxalate. Unless indicated elsewhere, these recommended doses can be used for the other embodiments of the invention. Individuals having mild-to-moderate Alzheimer's disease, according to this embodiment, are treated with the above-recommended daily doses for 24 weeks or more, preferably 36 weeks or more, even more preferably for one year or more, with the combination of R-flurbiprofen and SSRI. Desirably, the combination can be formulated in a single dosage form such as a tablet, capsule, or liquid for oral administration. Alternatively, the individual components of the combination (R-flurbiprofen and SSRI) can be administered separately, i.e., a tablet(s) of R-flurbiprofen and a tablet(s) having the S SRI.

In a preferred embodiment of the invention, a method for treating Alzheimer's disease is provided which involves administering to a patient an Aβ₄₂ lowering effective amount of a compound (i.e., R-flurbiprofen) and an SSRI.

In addition to using the R-NSAID and SSRI compounds, the invention includes using pharmaceutically acceptable prodrugs, pharmaceutically active metabolites, pharmaceutically acceptable esters, and pharmaceutically acceptable salts of such compounds.

Prodrugs and active metabolites of compound may be identified using routine techniques known in the art. See, e.g., Bertolini, G et al., J. Med. Chem., 40, 2011-2016 (1997); Shan, D. et al, J. Pharm. Sci., 86 (7), 756-767; Bagshawe K., Drug Dev. Res., 34, 220-230 (1995); Bodor N; Advance in Drug Res., 13, 224-331 (1984); Bundgaard, H., Design of Prodrugs (Elsevier Press 1985); and Larsen, I. K., Design and Application of Prodrugs, Drug Design and Development (Krogsgaard-Larsen et al., eds., Harwood Academic Publishers, 1991).

In addition, derivatives of R-flurbiprofen having the following formulae I and II can also be used in lieu of or in addition to, R-flurbiprofen:

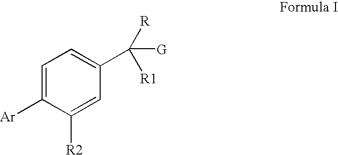

wherein:
R and R₁ are the same and are selected from the group of linear or branched C₁-C₄ alkyl; otherwise they form a 3 to 6 carbon atoms ring with the carbon atom to which they are linked;
G is: (1) a COOR″ group wherein R″ is H, linear or branched C₁-C₄ alkyl, C₃-C₆ cycloalkyl or ascorbyl; (2) a CONH₂ or a CONHSO₂R′″ group wherein R′″ is linear or branched C₁-C₄ alkyl or C₃-C₆ cycloalkyl; or (3) a tetrazolyl residue;
R₂ is H, CF₃, OCF₃ or a halogen selected from the group of F, Cl, Br, I, preferably fluorine.
   Ar is a group of formula 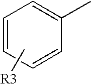
   wherein R₃ represents one or more groups independently selected from:
(1) halogen as previously defined; CF₃; C₃-C₈ cycloalkyl optionally substituted with one or more C₁-C₄ alkyl and/or oxo groups; CH═CH₂; CN; CH₂OH ; methylendioxy or ethylendioxy; NO₂;
(2) phenyl optionally substituted with one or more of the following groups: halogen; CF₃; OCF₃; OH; linear or branched C1-C₄ alkyl; a saturated heterocycle with at least 4 carbon atoms and at least 1 heteroatom; C₃-C8 cycloalkyl in turn optionally substituted with one or more of the following groups linear or branched C₁-C₄ alkyl, CF₃ or OH;
(3) OR₄ or NHCOR₄ wherein R₄ is CF₃, linear or branched C₂-C₆ alkenyl or alkynyl; benzyl; phenyl optionally substituted with one or more of the following groups: halogen, CF₃, OCF₃, OH, linear or branched C₁-C₄ alkyl; a saturated heterocycle with at least 4 carbon atoms and at least 1 heteroatom; C₃-C₈ cycloalkyl in turn optionally substituted with one or more of the following groups: linear or branched C₁-C₄ alkyl, CF₃ or OH;
(4) SR₅, SO₂R₅ or COR₅ wherein R₅ is linear or branched C₁-C₆ alkyl; otherwise Ar is a heterocycle ring selected from the group of thiophene, benzothiophene, dibenzothiophene, thianthrene, pyrrole, pyrazole, furan, BENZOFURAN, dibenzofuran, indole, isoindole, benzofurane, imidazole, benzoimidazole, oxazole, isoxazole, benzoxazole, thiazole, pyridine, pyrimidine, pyrazine, pyridazine, quinoline, isoquinoline, quinazoline, quinoxaline, cinnoline, pyrazole, pyran, benzopyran, pyrrolizine, phtalazine, 1,5-naphthyridine, 1,3-dioxole, 1,3-benzodioxole, optionally substituted with one or more groups R₃ as defined above; pharmaceutically acceptable salts and esters thereof.

Examples of such compounds of Formula I include 2-methyl-2 (2-fluoro-4′-trifluoromethylbiphen-4-yl) propionic acid; 2-methyl-2(2-fluoro-4′cyclohexyl biphen-4-yl) propionic acid; 1-(2-fluoro-4′-trifluoromethylbiphenyl-4-yl)cyclopropanecarboxylic acid; 1-(4′-cyclohexyl-2-fluorobiphenyl-4-yl)cyclopropanecarboxylic acid; 1-(4′-benzyloxy-2-fluorobiphenyl-4-yl)cyclopropanecarboxylic acid; 1-(2-fluoro-4′-isopropyloxybiphenyl-4-yl)cyclopropanecarboxylic acid; 1-(2-fluoro-3′-trifluoromethoxybiphenyl-4-yl)cyclopropanecarboxylic acid; 1-(2-fluoro-4′-trifluoromethoxybiphenyl-4-yl)cyclopropanecarboxylic acid; 1-(2-fluoro-3′-trifluoromethylbiphenyl-4-yl)cyclopropanecarboxylic acid; 1-(4′-cyclopentyl-2-fluorobiphenyl-4-yl) cyclopropanecarboxylic acid; 1-(4′-cycloheptyl-2-fluorobiphenyl-4-yl) cyclopropanecarboxylic acid; 1-(2′-cyclohexyl-2-fluorobiphenyl-4-yl) cyclopropanecarboxylic acid; 1-(2-fluoro-4′-hydroxybiphenyl-4-yl) cyclopropanecarboxylic acid; 1-[2-fluoro-4′-(tetrahydropyran-4-yloxy)biphenyl-4-yl ]-cyclopropane-carboxylic acid; 1-(2,3′,4′-trifluorobiphenyl-4-yl) cyclopropanecarboxylic acid; 1-(3′,4′-dichloro-2-fluorobiphenyl-4-yl) cyclopropanecarboxylic acid; 1-(3′,5′-dichloro-2-fluorobiphenyl-4-yl) cyclopropanecarboxylic acid; 1-(3′-chloro-2,4′-difluorobiphenyl-4-yl) cyclopropanecarboxylic acid; 1-(4-benzo [b]thiophen-3-yl-3-fluorophenyl)cyclopropanecarboxylic acid; 1-(2-fluoro-4′-prop-2-inyloxy-biphenyl-4-yl)-cyclopropanecarboxylic acid; 1-(4′-cyclohexyloxy-2-fluoro-biphenyl-4-yl)-cyclopropanecarboxylic acid; 1-[2-fluoro-4′-(tetrahydropyran-4-yl)-biphenyl-4-yl]-cyclopropanecarboxylic acid; 1-[2-fluoro-4′-(4-oxo-cyclohexyl)-biphenyl-4-yl]-cyclopropanecarboxylic acid; 2-(2″-fluoro-4-hydroxy-[1,1′,4′,1″]tert-phenyl-4″-yl) -cyclopropanecarboxylic acid; 1-[4′-(4,4-dimethylcyclohexyl)-2-fluoro[1,1′-biphenyl]-4-yl]-cyclopropane-carboxylic acid; 1-[2-fluoro-4′-[[4-(trifluoromethyl)benzoyl]amino][1,1′-biphenyl]-4-yl]-cyclopropanecarboxylic acid; 1-[2-fluoro-4′-[[4-(trifluoromethyl) cyclohexyl]oxy][1,1′-biphenyl]-4-yl]-cyclopropanecarboxylic acid; 1 -[2-fluoro-4′-[(3,3,5,5-tetramethylcyclohexyl)oxy][1,1′-biphenyl]-4-yl]-cyclopropanecarboxylic acid; 1-[4′-[(4,4-dimethylcyclohexyl)oxy]-2-fluoro[1,1′-biphenyl ]-4-yl]-cyclopropanecarboxylic acid; 1-(2,3′,4″-trifluoro[1,1′:4′,1″-tert-phenyl]-4-yl)-cyclopropanecarboxylic acid; 1-(2,2′,4″-trifluoro [1,1′:4′,″-tert-phenyl]-4-yl)-cyclopropanecarboxylic acid; 1-(2,3′-difluoro-4″-hydroxy [1,1′:4′,1″-tert-phenyl]4-yl)-cyclopropane-carboxylic acid; 1-(2,2′-difluoro-4″-hydroxy [1,1′:4′, 1″-tert-phenyl]-4-yl)-cyclopropane-carboxylic acid; 2-(2-fluoro-3′,5′-bis (chloro) biphen-4-yl) propionic acid amide. 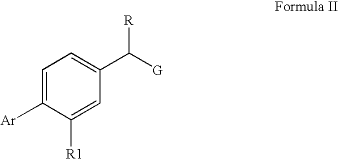
wherein:
R is linear or branched C₁-C₄ alkyl;
G is: (1) a COOR″ group wherein R″ is H, linear or branched C₁-C₄ alkyl, C₃-C₆ cycloalkyl or ascorbyl; (2) a tetrazolyl residue; R₁ is CF₃, OCF₃ or a halogen selected from the group of F, Cl, Br, I, preferably fluorine.
   Ar is A group of formula 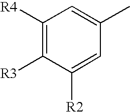
   wherein R₂; R₃ and R₄ are independently selected from the group of: H; CF₃; optionally substituted C₃₋₅ cycloalkyl; optionally substituted phenyl; and enantiomers and pharmaceutically acceptable salts thereof.

As shown in PCT Publication Nos. WO 2004/074232 and WO 2004/073705, the compounds of Formulae I and II are capable of inhibiting Aβ₄₂ release while substantially free of Cox inhibition activities. Thus are expected to have similar utilities and functions as R-flurbiprofen in the compositions and methods of the present invention.

In preferred embodiments, a dosage having R-flurbiprofen in an amount of about 400 mg to about 800 mg per dose is included in the combination of the present invention. The dose can be provided twice daily, in a single or multiple dosage units (i.e., tablets or capsules) having about 350 mg R-flurbiprofen, 400 mg R-flurbiprofen, 450 mg R-flurbiprofen, 500 mg R-flurbiprofen, 550 mg R-flurbiprofen, 600 mg R-flurbiprofen, 650 mg R-flurbiprofen, 700 mg R-flurbiprofen, 750 mg R-flurbiprofen, 800 mg R-flurbiprofen, or 850 mg R-flurbiprofen or a pharmaceutically acceptable salt or ester thereof. For example, the dosage unit is 400 mg; thus, a preferred composition of the invention comprises 400 mg R-flurbiprofen and a selective serotonin reuptake inhibition effective amount of a SSRI, and a carrier or vehicle suitable for oral administration, e.g., in tablets or capsules. Another preferred dose is 800 mg of R-flurbiprofen, and a preferred composition of the invention comprises 800 mg R-flurbiprofen and a selective serotonin reuptake inhibition effective amount of a SSRI, and a carrier or vehicle suitable for oral administration, e.g., in tablets or capsules. Preferably, the compositions are substantially free of S-flurbiprofen. In one preferred embodiment, the method and composition are applied such that R-flurbiprofen is administered at 400 mg per dose every 12 hours (i.e., twice daily), separately or along with an SSRI. In another preferred embodiment, the method and composition are applied such that a R-flurbiprofen is administered at 800 mg per dose every 12 hours (i.e., twice daily), separately or along with an SSRI.

Oral administration of a dose, twice daily for at least 4 months, preferably 8 months, and more preferably 1 year, can provide an improvement or lessening of decline in cognitive function, biochemical disease marker progression, and/or plaque pathology.

Desirably, the R-flurbiprofen-containing compositions of the invention are substantially free of the S-stereoisomer of flurbiprofen. In one aspect, substantially free of the S-stereoisomer means at least 90% by weight R-flurbiprofen to 10% by weight or less of S-flurbiprofen of the total flurbiprofen (S+R flurbiprofen) in said pharmaceutical composition. In another aspect, the ratio between R-flurbiprofen and S-flurbiprofen in the pharmaceutical composition is at least 95% by weight to 5% by weight. More preferably, the ratio is at least 99% by weight R-flurbiprofen to 1% by weight or less of S-flurbiprofen in the pharmaceutical composition. Even more preferably, the ratio is at least 99.9% by weight R-flurbiprofen to 0.1% by weight or less of S-flurbiprofen in the pharmaceutical composition. In one aspect, a preferred dosage form is a tablet. In another aspect, a preferred dosage form is a capsule. In other aspects, the composition provides an improvement or lessening in decline in biochemical disease marker progression, plaque pathology, quality of life indicators or combinations of any disease parameters.

In preferred embodiments, in the R-flurbiprofen-containing compositions and combination treatment methods of the present invention, R-flurbiprofen or a pharmaceutically acceptable salt or ester or prodrug thereof is administered in an amount sufficient to result in a plasma R-flurbiprofen Cₘₐₓ of about 20 to about 150 μg per mL, and wherein said individual is known to have, or is suspected of having, AD or MCI. In a more specific embodiment, said plasma R-flurbiprofen Cₘₐₓ is from about 30 to about 95 μg per mL. In another more specific embodiment, said Cₘₐₓ is from about 40 to about 80 μg per mL. In another embodiment, said Cₘₐₓ is between about 100 and about 600 μM. In a more specific embodiment, said plasma Cₘₐₓ is from about 150 to about 380 μM. In another more specific eembodiment, said Cₘₐₓ is from about 170 to about 240 μM. In a specific, preferred embodiment, said individual has mild to moderate AD or MCI.

In another embodiment, R-flurbiprofen or a pharmaceutically acceptable salt or ester thereof is administered in an amount sufficient to result in a cerebrospinal fluid R-flurbiprofen Cₘₐₓ of about 0.05 to about 7.5 μg per mL, and wherein said individual is known to have, or is suspected of having, AD or MCI. In another embodiment, said Cₘₐₓ is from about 0.08 to about 4.5 μg per mL. In another embodiment, the R-flurbiprofen or a pharmaceutically acceptable salt or ester thereof is administered in an amount sufficient to result in a cerebrospinal fluid R-flurbiprofen Cₘₐₓ of about 2 to 30 μM; from about 3.2 μM to about 20 μM; or from about 4 μM to about 12 μM.

The time to achieve plasma R-flurbiprofen Cₘₐₓ will depend upon the individual to be treated, but is preferably between 0.70 to 3.75 hours. In various embodiments, the tₘₐₓ (time to Cₘₐₓ ) is from about 0.75 to 2.00 hours, or is from about 0.75 hour to about 1.75 hours. For example, tₘₐₓ can be about 2 hours after administration. Preferably, the t_{1/2} (half-life) is from about 3.75 to about 8.5 hours.

Somewhat more time is expected to achieve a cerebrospinal fluid Cₘₐₓ; however, this Cₘₐₓ is expected to be achieved between about 1 hour and about 6 hours after administration of a dose of R-flurbiprofen according to the invention.

R-flurbiprofen levels in the plasma or in the cerebrospinal fluid may be assessed by any art-accepted method. Determination of the concentration of R-flurbiprofen in cerebrospinal fluid may be accomplished as follows. Cerebrospinal fluid containing flurbiprofen and an internal standard, for example, flurbiprofen-D₃, is mixed with mobile phase and centrifuged. The supernatant is then transferred to a 96-well block and an aliquot of extract is injected onto a Micromass Ultima LC-MS-MS equipped with an enantio-selective column. Peak area of the m/z 243→199 flurbiprofen product ion is measured against the peak area of the m/z 246→202 flurbiprofen-D₃ internal standard product ion. Quantification may be performed using a weighted (1/x²) linear least squares regression analysis for each enantiomer generated from fortified plasma standards prepared in bulk and frozen.

The plasma half-life will also depend upon the individual to be treated. Preferably, the plasma half-life of R-flurbiprofen is from about 3.75 to about 8.5 hours. Preferably, administration of a single dose to a fasting subject provides an AUC (area under curve of concentration versus time; total drug exposure) of R-flurbiprofen of from about 200 hr·μg/mL to about 600 hr·μg/mL. Also preferably, the R-flurbiprofen in the compositions and methods of the present invention is such that in repeating administrations an AUC₁₂ (area under curve of concentration in a 12-hour window, i.e., total drug exposure in a 12-hour window) is from about 200 hr·μg/mL to about 450 hr·μg/mL. Thus, in one embodiment, a composition of the present invention is administered to an individual having one or more indications of Alzheimer's disease or MCI, to achieve a plasma concentration in said individual of R-flurbiprofen of between 30 and 95 μg per mL by no more than 3.75 hours after administration. In a specific embodiment, said plasma concentration is achieved within 1.75 hours after administration. In another specific embodiment, said plasma concentration is achieved between 0.75 hours and 3.75 hours after administration. In another specific embodiment, said plasma concentration is between 40 and 80 μg per mL. In another specific embodiment, said individual is an individual that has been diagnoses having mild to moderate Alzheimer's disease or MCI or that would be diagnosed as having mild to moderate Alzheimer's disease or MCI.

In one embodiment, the R-flurbiprofen-containing compositions of the present invention are administered for a method of administering R-flurbiprofen to an individual, wherein said R-flurbiprofen is administered in an amount sufficient to result in a plasma R-flurbiprofen Cₘₐₓ of about 35 to about 50 μg per mL, and wherein said individual is known to have, or is suspected of having, AD or MCI. In a more specific embodiment, said plasma Cₘₐₓ is from about 38 to about 48 μg per mL. In another more specific embodiment, said Cₘₐₓ is from about 39 to about 46 μg per mL. In another embodiment, the invention provides for a method of administering R-flurbiprofen to an individual, wherein said R-flurbiprofen is administered in an amount sufficient to result in a plasma Cₘₐₓ of about 45 to about 58 μg per mL, and wherein said individual is known to have, or is suspected of having, AD. In a more specific embodiment, said plasma Cₘₐₓ is from about 47 to about 56 μg per mL. In a more specific embodiment, said plasma Cₘₐₓ is from about 48 to about 55 μg per mL. In a specific, preferred embodiment, said individual has mild to moderate AD. In another specific, preferred embodiment, said individual has MCI.

In another embodiment, the time to achieve plasma R-flurbiprofen Cₘₐₓ will depend upon the individual to be treated, but is preferably between 0.70 to 3.00 hours. In various preferred embodiments, the R-flurbiprofen tₘₐₓ (time to Cₘₐₓ ) is from about 1.0 to 2.5 hours, or is from about 1.25 hour to about 2 hours, or is from about 1.40 to about 1.75 hours. Preferably, the t_{1/2} (half-life) is from about 6.00 to about 10.0 hours; from about 6.5 to about 9.5 hours; and from about 7 to about 9 hours. Preferably the R-flurbiprofen AUC (area under the curve; total drug exposure) is from about 350 (hr*μg/mL) to 750 (hr*μg/mL); is from about 400 (hr*μg/mL) to 650 (hr*μg/mL); or is from about 450 (hr*μg/mL) to 700 (hr*μg/mL). In a specific, preferred embodiment, said individual has mild to moderate AD. In another specific, preferred embodiment, said individual has MCI.

In yet another embodiment, the time to achieve plasma R-flurbiprofen Cₘₐₓ will depend upon the individual to be treated, but is preferably between 0.25 to 2.00 hours. In various preferred embodiments, the R-flurbiprofen tₘₐₓ (time to Cₘₐₓ) is from about 0.25 to 1.75 hours, or is from about 0.50 hour to about 1.75 hours, or is from about 0.5 to about 1.25 hours. Preferably, the R-flurbiprofen t_{1/2} (half-life) is from about 3.5 to about 8.5 hours; more preferably from about 4.0 to about 8.0 hours; and more preferably from about 4.8 to about 7.5 hours. Preferably the R-flurbiprofen AUC (area under the curve; total drug exposure) is from about 250 (hr*μg/mL) to 700 (hr*μg/mL); is from about 300 (hr*μg/mL) to 650 (hr*μg/mL); or is from about 350 (hr*μg/mL) to 600 (hr*μg/mL). In a specific, preferred embodiment, said individual has mild to moderate AD. In another specific, preferred embodiment, said individual has MCI.

Preferably, the method and composition are applied such that R-flurbiprofen or a prodrug or salt or ester thereof is administered once every 12 hours (i.e., twice daily), separately or along with an SSRI, each administration being at a dosage sufficient to achieve the above pK profiles.

Alzheimer's Disease Diagnosis

An AD diagnosis can be made using any known method. Typically, AD is diagnosed using a combination of clinical and pathological assessments. For example, progression or severity of AD can be determined using Mini Mental State Examination (MMSE) as described by Mohs et al. Int Psychogeriatr 8:195-203 (1996); Alzheimer's Disease Assessment Scale-cognitive component (ADAS-cog) as described by Galasko et al. Alzheimer Dis Assoc Disord, 11 suppl 2:S33-9 (1997); the Alzheimer's Disease Cooperative Study Activities of Daily Living scale (ADCS-ADL) as described by McKhann et al. Neurology 34:939-944 (1984); and the NINCDS-ADRDA criteria as described by Folstein et al. J. Psychiatr. Res. 12:189-198 (1975). In addition, methods that allow for evaluating different regions of the brain and estimating plaque and tangle frequencies can be used. These methods are described by Braak et al. Acta Neuropathol 82:239-259 (1991); Khachaturian Arch. Neuro. 42:1097-1105 (1985); Mirra et al. (1991) Neurology 41:479-486; and Mirra et al. Arch Pathol Lab Med 117:132-144 (1993). Newer methods such as PET scan and Aβ-42 level determination may also be used.

Definitions

As used herein, the term “SSRI” refers to a class of pharmaceuticals known as selective serotonin reuptake inhibitors. SSRIs include, but are not limited to, fluoxetine, fluvoxamine, paroxetine, sertraline, citalopram, escitalopram oxalate, compounds described in U.S. Pat. No. 4,314,081, U.S. Pat. No. 4,085,225, U.S. Pat. No. 4,721,723, U.S. Pat. No. 4,536,518, U.S. Pat. No. 4,136,193, and U.S. Pat. No. 6,455,710 B1, all of which are hereby incorporated by reference in their entireties. The skilled artisan recognizes that when referring to the SSRIs herein, the reference is to the active ingredient and is not limited to one particular salt form. For example, the active ingredient of escitalopram oxalate is escitalopram. PROZAC® (fluoxetine) is commercially available from Eli Lilly and is (±)N-methyl-3-phenyl-3-[(α,α,α-trifluoro-p-tolyl)oxy]propylamine hydrochlordie and has an empirical formula of C₁₇H₁₈F₃NO.HCL. LUVOX® (Fluvoxamine) is commercially available from Pfizer and is 5-methoxy-4′-(trifluoromethyl)valerophenone-(E)-O-(2-aminoethyl)oxime maleate (1:1) and has the empirical formula C₁₅H₂₁O₂N₂F₃.C₄H₄O₄. PAXIL® (Paroxetine) is commercially available from Smithkline Beecham and is (−)-trans-4R-(4′-fluorophenyl)-3S-[(3′,4′-methylenedioxyphenoxy) methyl]piperdine hydrochloride hemihydrate and has the empirical formula C₁₉H₂₀FNO₃.HCl.1/2H₂O. ZOLOFT® (sertraline) is commercially available from Pfizer and is (1S-cis)-4-(3,4-dichlorophenyl)-1,2,3,4-tetrahydro-N-methyl-1-naphthalenamine hydrochloride and has the empirical formula of C₁₇H₁₇NCl₂.HCL. CELEXA® (citalopram) is commercially available from Forest Pharmaceuticals and is (+/−)-1-(3-dimethylaminopropyl)-1-(4-fluorophenyl)-1,3-dihydroisobenzofuran-5-carbonitrile HBr and has the empirical formula C₂₀H₂₂BrFN₂O.

As used herein, the term “unit dosage form” refers to a physically discrete unit, such as a capsule or tablet suitable as a unitary dosage for a human patient. Each unit contains a predetermined quantity of active ingredient(s), which was discovered or believed to produce the desired pharmacokinetic profile which yields the desired therapeutic effect. The dosage unit is composed of the active ingredient(s) in association with at least one pharmaceutically acceptable carrier, salt, excipient, or combination thereof.

As used herein, the term “dose” or “dosage” refers the amount of active ingredient that an individual takes or is administered at one time. For example, an 800 mg dose of a compound of R-NSAID refers to, in the case of a twice-daily dosage regimen, a situation where the individual takes 800 mg of a compound of R-NSAID twice a day, e.g., 800 mg in the morning and 800 mg in the evening. 800 mg of a compound of the R-NSAID dose can be divided into two or more dosage units, e.g., two 400 mg dosage units of R-NSAID in tablet form or two 400 mg dosage units of R-NSAID in capsule form. The dose can contain two active ingredients, i.e., R-flurbiprofen and one or more SSRIs.

“A pharmaceutically acceptable prodrug” is a compound that may be converted under physiological conditions or by solvolysis to the specified compound or to a pharmaceutically acceptable salt of such compound.

“A pharmaceutically active metabolite” is intended to mean a pharmacologically active product produced through metabolism in the body of a specified compound or salt thereof. Metabolites of a compound may be identified using routine techniques known in the art and their activities determined using tests such as those described herein.

“A pharmaceutically acceptable salt” is intended to mean a salt that retains the biological effectiveness of the free acids and bases of the specified compound and that is not biologically or otherwise undesirable. A compound for use in the invention may possess a sufficiently acidic, a sufficiently basic, or both fanctional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrophosphates, dihydrophosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, flimarates, maleates, butyne-1,4dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, gamma-hydroxybutyrates, glycollates, tartrates, methane-sulfonates, propanesulfonates, naphthalene-1-sulfonates, naphthalene-2-sulfonates, and mandelates.

As used herein, the phrase “treating . . . with . . . (a compound)” or a paraphrase or equivalent thereof means either administering the compound to a patient, or administering to a patient the compound or another agent to cause the presence or formation of the compound inside the patient.

Additional Combination Therapy

The invention further provides additional combination therapy strategies for treating neurodegenerative disorders such as Alzheimer's disease, mild cognitive impairment (MCI), and dementia. According to this aspect of the invention, an individual in need of treatment is administered an effective amount of an R-NSAID (e.g., R-flurbiprofen), at least one SSRI (such as fluoxetine, fluvoxamine, paroxetine, sertraline, citalopram, and escitalopram oxalate) and at least one compound selected from the group consisting of NSAIDs, COX-2 inhibitors (cyclooxygenase-2), β-secretase inhibitors, γ-secretase inhibitors, acetylcholine esterase inhibitors, NMDA antagonists (i.e., memantine), and GABA-A alpha inverse agonist (see WO 00/27382, WO 96/25948, WO 98/50385 which are herein incorporated by reference in there entireties). Preferred acetylcholine esterase inhibitors include tacrine, donepezil, rivastigmine, and galantamine. Preferred NMDA receptor antagonists for combination therapy are memantine, adamantane, amantadine, an adamantane derivative, dextromethorphan, dextrorphan, dizocilpine, ibogaine, ketamine, and remacemide. The combination therapy of the invention is thought to provide a synergistic effect in reducing Aβ₄₂ levels and is surprisingly thought to be especially effective for treating and preventing neurodegenerative disorders including Alzheimer's disease, dementia, and MCI. The invention further encompasses compositions comprising the combination of active ingredients of this aspect of the invention.

According to another aspect of the invention, an individual in need of such treatment is administered an effective amount of R-flurbiprofen, at least one SSRI such as fluoxetine, fluvoxamine, paroxetine, sertraline, citalopram, and escitalopram oxalate, and at least one NSAID. According to a preferred aspect of this embodiment the NSAID is selected from the group consisting of 5,5-dimethyl-3-(3-fluorophenyl)-4-(4-methylsulfonyl)phenyl-2(5H)-furanone, 5,5-dimethyl-3-isopropyloxy-4-(4′-methylsulfonylphenyl)-2(5H)-furanone, resveratrol, flufemic acid, meclofenamic acid, fenoprofen, carprofen, ibuprofen, ketoprofen, sulindac, indomethacin, naproxen, etolodac, tiaprofenic, suprofen, ketorolac, pirprofen, indoprofen, benoxaprofen, oxaprozin, diflunisal, and nabumetone.

The treatment regime used in the combination therapy can involve administration of a composition comprising the combination of active ingredients, the concomitant administration of separate compositions, each comprising at least one active ingredient.

Furthermore, the administration of the active ingredients can be performed at different times and/or different routes. For example, a composition having one active ingredient can be administered in the morning, and a composition having the other active ingredients can be administered in the evening. Another example would involve the administration of a composition having two active ingredients orally while the third active ingredient is administered intravenously.

Preparation of the Compounds of the Invention

The compounds of the invention can be prepared by a variety of art known procedures. In one aspect, the R-NSAID employed in the compositions and methods disclosed herein can be selected from the group consisting of selected R-flurbiprofen, R-ibuprofen, R-ketoprofen, R-naproxen, R-tiaprofenic acid, R-suprofen, R-carprofen, R-pirprofen, R-indoprofen, and R-benoxaprofen. The R-NSAID can also be a cyclized derivative of an arylpropionic acid, such as R-ketorolac, or an arylacetic acid, such as R-etodolac. Descriptions of specific NSAIDs and their preparation can be found in various publications. R-Ibuprofen is described in U.S. Pat. No. 6,255,347. Ketoprofen is described in U.S. Pat. No. 3,641,127. Flurbiprofen is described in U.S. Pat. No. 3,755,427. Ketorolac is described in U.S. Pat. No. 4,089,969.

A large number of the NSAIDs useful according to the invention are commercially available either in the form of racemic mixtures or as optically pure enantiomers. In all cases racemic mixtures contain equal amounts of the R- and S-isomers of the NSAID are provided. For example, the following racemates can be obtained through Sigma Chemical Co.: ketoprofen, flurbiprofen, etodolac, suprofen, carprofen, indoprofen and benoxaprofen. Naproxen, marketed as the S-isomer only, is also available from this source. Additionally, many commercial sources exist for the stereospecific R-isomers of many NSAIDs. R-ketoprofen, R-flurbiprofen and R-ketorolac, for example, are available through Sepracor, Inc.; R-naproxen can be obtained as the sodium salt through Sigma Chemical Co.; R-etodolac is available from Wyeth-Ayerst; R-tiaprofenic acid is available through Roussel (France, Canada, Switzerland, Spain, Denmark, Italy); R-suprofen is manufactured by McNiel Pharmaceuticals; R-carprofen is available from Roche; R-pirprofen is available through Ciba (France, Belgium, Denmark); R-indoprofen can be obtained through Carlo Elba (Italy, U.K.); and R-benoxaprofen is manufactured by Eli Lilly Co. SSRIs for use in this invention are any including: fluoxetine marketed under the trademark PROZAC by Dista & Eli Lilly, and described in U.S. Pat. No. 4,085,225; fluvoxamine, marketed under the trademark LUVUX by Solvay, and described in U.S. Pat. No. 4,085,225; paroxetine, marketed under the trademark PAXIL by Smithkline Beecham and described in U.S. Pat. No. 4,721,723; sertraline, marketed under the trademark ZOLOFT by Pfizer, and described in U.S. Pat. No. 4,536,518; citalopram, marketed under the trademark CELEXA by Forest Pharmaceuticals, and described in U.S. Pat. No. 4,650,884; and escitalopram oxalate, marketed under the trademark LEXAPRO by Forest Pharmaceuticals, and described in U.S. Pat. No RE34,712. All of the patents referenced in this section are hereby incorporated by reference in their entireties.

Dosages, Formulations and Route of Administration

The active compounds of this invention are typically administered in combination with a pharmaceutically acceptable carrier through any appropriate routes such as parenteral, oral, or topical administration, in a therapeutically (or prophylactically) effective amount according to the methods set forth above. A preferred route of administration for use in the invention is oral administration.

Generally, the toxicity profile and therapeutic efficacy of the therapeutic agents can be determined by standard pharmaceutical procedures in suitable cell models or animal models. As is known in the art, the LD₅₀represents the dose lethal to about 50% of a tested population. The ED₅₀ is a parameter indicating the dose therapeutically effective in about 50% of a tested population. Both LD₅₀ and ED₅₀ can be determined in cell models and animal models. In addition, the IC₅₀ may also be obtained in cell models and animal models, which stands for the circulating plasma concentration that is effective in achieving about 50% of the maximal inhibition of the symptoms of a disease or disorder. Such data may be used in designing a dosage range for clinical trials in humans. Typically, as will be apparent to skilled artisans, the dosage range for human use should be designed such that the range centers around the ED₅₀ and/or IC₅₀, but remains significantly below the LD₅₀ dosage level, as determined from cell or animal models.

Typically, the compounds and compositions for use in the invention can be effective at an amount of from about 0.05 mg to about 4000 mg per day, preferably from about 0.1 mg to about 2000 mg per day. However, the amount can vary with the body weight of the patient treated and the state of disease conditions. The active ingredient may be administered at once, or may be divided into a number of smaller doses to be administered at predetermined intervals of time. The EC₅₀ values discussed previously can desirably be used to identify specific pro-apoptotic compounds and compositions that can be used within predetermined, desirable dosage ranges.

In the case of combination therapy, a therapeutically effective amount of another therapeutic compound can be administered in a separate pharmaceutical composition, or alternatively included in the pharmaceutical composition according to the present invention. The pharmacology and toxicology of other therapeutic compositions are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in the art can be equally applicable in the present invention.

It should be understood that the dosage ranges set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can also be adjusted as the various factors change over time.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacterial agents, surfactants, and antioxidants can all be included. For example, useful components include sodium chloride, acetate, citrate or phosphate buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown, et al., Annual Review of Medicine, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the active compounds. Hydrogels are generally known in the art. They are typically made by crosslinking high molecular weight biocompatible polymers into a network that swells in water to form a gel like material. Preferably, hydrogels are biodegradable or biosorbable. For purposes of this invention, hydrogels made of polyethylene glycols, collagen, or poly(glycolic-co-L-lactic acid) may be useful. See, e.g., Phillips et al., J. Pharmaceut. Sci. 73:1718-1720 (1984).

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

Soft gelatin capsules can be prepared in which capsules contain a mixture of the active ingredient and vegetable oil or non-aqueous, water miscible materials such as, for example, polyethylene glycol and the like. Hard gelatin capsules may contain granules of the active ingredient in combination with a solid, pulverulent carrier, such as, for example, lactose, saccharose, sorbitol, mannitol, potato starch, corn starch, amylopectin, cellulose derivatives, or gelatin.

Tablets for oral use are typically prepared in the following manner, although other techniques may be employed. The solid substances are ground or sieved to a desired particle size, and the binding agent is homogenized and suspended in a suitable solvent. The active ingredient and auxiliary agents are mixed with the binding agent solution. The resulting mixture is moistened to form a uniform suspension. The moistening typically causes the particles to aggregate slightly, and the resulting mass is gently pressed through a stainless steel sieve having a desired size. The layers of the mixture are then dried in controlled drying units for determined length of time to achieve a desired particle size and consistency. The granules of the dried mixture are gently sieved to remove any powder. To this mixture, disintegrating, anti-friction, and anti-adhesive agents are added. Finally, the mixture is pressed into tablets using a machine with the appropriate punches and dies to obtain the desired tablet size. The operating parameters of the machine may be selected by the skilled artisan.

If the compound for use in the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound for use in the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium. These substituents may optionally be further substituted with a substituent selected from such groups.

### EXAMPLES

### Example 1

### Co-Formulation of R-flurbiprofen with SSRIs

R-Flurbiprofen Fluoxetine Tablets

| Ingredient | Amount | |
|---|---|---|
| R-Flurbiprofen | 400 | mg |
| Microcrystalline Cellulose | 392 | mg |
| Colloidal Silicon Dioxide | 4 | mg |
| Magnesium Stearate | 4 | mg |
| Fluoxetine | 40 | mg |

R-Flurbiprofen Fluvoxamine Tablets

| Ingredient | Amount | |
|---|---|---|
| R-Flurbiprofen | 400 | mg |
| Microcrystalline Cellulose | 392 | mg |
| Colloidal Silicon Dioxide | 4 | mg |
| Magnesium Stearate | 4 | mg |
| Fluvoxamine | 100 | mg |

R-Flurbiprofen Paroxetine Tablets

| Ingredient | Amount | |
|---|---|---|
| R-Flurbiprofen | 400 | mg |
| Microcrystalline Cellulose | 392 | mg |
| Colloidal Silicon Dioxide | 4 | mg |
| Magnesium Stearate | 4 | mg |
| Paroxetine | 40 | mg |

R-Flurbiprofen Sertraline Tablets

| Ingredient | Amount | |
|---|---|---|
| R-Flurbiprofen | 400 | mg |
| Microcrystalline Cellulose | 392 | mg |
| Colloidal Silicon Dioxide | 4 | mg |
| Magnesium Stearate | 4 | mg |
| Sertraline | 100 | mg |

R-Flurbiprofen Citalopram Tablets

| Ingredient | Amount | |
|---|---|---|
| R-Flurbiprofen | 400 | mg |
| Microcrystalline Cellulose | 392 | mg |
| Colloidal Silicon Dioxide | 4 | mg |
| Magnesium Stearate | 4 | mg |
| Citalopram | 30 | mg |

R-Flurbiprofen Escitalopram Oxalate Tablets

| Ingredient | Amount | |
|---|---|---|
| R-Flurbiprofen | 400 | mg |
| Microcrystalline Cellulose | 392 | mg |
| Colloidal Silicon Dioxide | 4 | mg |
| Magnesium Stearate | 4 | mg |
| Escitalopram Oxalate | 10 | mg |

The tablets are prepared using art known procedures and the amounts ingredients listed above can be modified to obtain an improved formulation.

### Example 2

### Clinical Investigation of the Combination of R-flurbiprofen and a SSRI for Alzheimer's Disease

According to this example, R-flurbiprofen in combination with an SSRI is examined for its actions in healthy subjects as well as subjects with mild to moderate

Alzheimer's disease (AD). Evaluation of a R-flurbiprofen and an SSRI for treating Alzheimer's is accomplished in a three-group parallel design; each group having 53 subjects for a total of 159 subjects. Subjects are treated with R-flurbiprofen and a SSRI (e.g., sertraline) or a matching placebo twice a day for forty-eight weeks.

Test AD subjects are selected based on the following criteria: Subjects (1) have a diagnosis of dementia according to the DSM IV (TR) and meets the NINCDS-ADRDA (McKhann et al. Neurology 34:939-944 (1984)) criteria for probable Alzheimer's disease, (2) have CT or MRI since onset of memory impairment demonstrating absence of clinically significant focal adhesion, (3) have MMSE (Mohs et al. Int Psychogeriatr 8:195-203 (1996)) score≧15 and ≦26, (4) have a modified Hachinski Ischaemic score<4, (5) age≧45 years and living in the community at the time of enrollment, (6) signed patient informed consent form and willing/able to attend for duration of study, (7) read and understand English, six years of education or work history sufficient to exclude mental retardation. Subjects can have no unforeseen aspirin use other than for cardioprotective therapy (e.g.,<325 mg aspirin/day). Subjects taking acetylcholinesterase inhibitors may be enrolled as long as they have been on a stable treatment dose for at least three months. Subjects must have a reliable English speaking caregiver or informant to accompany the subject for clinic visits and be prepared to supervise medication.

Subjects are excluded according to the following criteria: treatment with memantine in past 4 weeks, current evidence or history in the last 2 years of epilepsy, focal brain lesion, head injury with loss of consciousness and or immediate confusion after the injury, or DSM-IV criteria for major psychiatric disorder including psychosis, major depression, bipolar disorder, alcohol or substance abuse, history of hypersensitivity to NSAIDS including COX-2 inhibitors, chronic use of NSAIDs at any dose more than 7 days per month for the two months prior to Study day 1, history of upper GI bleeding requiring treatment with the past 3 years, documented evidence of active gastric or duodenal ulcer disease within the past three months, history of NSAID-associated ulcers, history of, or evidence of active malignancy, except basal cell carcinoma and squamous cell carcinoma of the skin within the 24 months prior to entry, chronic or acute renal, hepatic, or metabolic disorder or any other condition, which in the Investigator's opinion, might preclude study participation, use of any investigational therapy within 30 days, or 5 half-lives whichever is longer, prior to screening, major surgery within 12 weeks prior to Study Day 1, patients with uncontrolled cardiac conditions (New York Heart Association Class III or IV), anticoagulant therapy such as warfarin with 12 weeks prior to randomization, treatment with any CYP2C9 inhibitor within a two-week period prior to randomization (examples include amiodarone/Cordarone®, fluconazole/Diflucan®, fluvoxamine/Luvox®, isoniazid/INH®, miconazole/Monistat®, phenylbutazone/Butazolidone®, probenicid/Benemid®, sulfamethoxazole/Gantanol®, sulfaphenazole, teniposide/Vumon®, trimethoprim/Bactrim®, zafirlukast/Accolate®; danshen (Salvia miltiorrhiza); Lycium barbarun.

Therapeutic Endpoints

The primary efficacy endpoint is the rate of decline in the ADAS-cog score based on either a slope calculated for each patient or on a Generalized Estimating Equations (GEE) model. Secondary efficacy endpoints can include scores on the CIBIC+, NPI, ADCS-ADL, and CDR sum of boxes. Efficacy analyses for primary and secondary endpoints can include the baseline score as a covariate, and will also include a term for the stratification variable: use or nonuse of acetylcholinesterase inhibitor baseline. A modified intent to treat approach can be used in which all randomized subjects who receive any study treatment and have post-baseline efficacy assessment can be included in the intent to treat population using a last value carried forward approach. A per protocol analysis population can include all subjects in the intent to treat population who did not have any major protocol violations.

Subjects consist of men and women, ages 60-85, who are diagnosed with probable AD using the National Institute of Neurologic Communicative Disorders and Stroke-Alzheimer's Disease and Related Disorders Association (NINCDS-ADRDA) test (McKhann et al. Neurology 34:939-944 (1984)) or have mild to moderate dementia as determined by the Mini-Mental State Examination (MMSE, Mohs et al. Int Psychogeriatr 8:195-203 (1996)). MMSE scores in the range of 15-25 indicate mild to moderate dementia. AD subjects have caregivers that can ensure compliance with medication regimens and with study visits and procedures.

Control subjects consist of men and women ages 60-80 that lack significant cognitive or functional complaints, or depression as determined by the Geriatric Depression Scale (GDS), and have MMSE scores in the range of 27-30. Control subjects have the same general requirements as AD subjects with the exception that caregivers are not required. Both AD subjects and control subjects have good general health, i.e., subjects do not have serious or life-threatening comorbid conditions.

Subjects who have medically active major inflammatory comorbid condition(s) such as rheumatoid arthritis, or those who have peptic ulcer, gastro-intestinal bleeding, or intolerance of NSAIDs in the past are excluded from the study. Those who have contra-indications to lumbar puncture, such as severe lumbar spine degeneration, sepsis in the region of the lumbar spine, or a bleeding disorder are excluded from participation in the study. In addition, subjects who currently or recently use medications such as NSAIDs, prednisone, or immunosuppressive medications such as cyclophosphamide that could interfere with the study are excluded. Recently is defined as within one month before undergoing the baseline visit (see next paragraph). Subjects undergoing acetylcholinsterase inhibitor (AChE-I) treatments for AD are not excluded if these subjects have been on stable doses for at least four weeks. Similarly, AD subjects taking antioxidants such as vitamin E, vitamin C, or Gingko biloba are not excluded if they have been on stable doses for at least four weeks. Subjects who use NSAIDs or aspirin on a regular basis are excluded. If needed, analgesics such as paracetamol (Tylenol) are provided during the fourteen-day study.

The study procedure consists of three in-clinic visits: an initial screening visit, a baseline visit, and a follow-up visit at fourteen days. During the screening visit, information needed to assess eligibility is obtained and MMSE is administered.

During the baseline visit, which takes place within two weeks of the screening visit, physical examinations and lumbar punctures are performed. Blood samples are drawn for laboratory tests such as APO-E genotyping and for plasma preparation. At this time, subjects or caregivers, in the case of AD subjects, are given a supply of study R-flurbiprofen and an SSRI, along with instructions about timing of doses and potential adverse effects. (For AD subjects, caregivers are required to accompany subjects to each visit, and are responsible for monitoring and supervising administration of study drugs). A calendar is provided on which times of medications and potential adverse symptoms are recorded.

The treatment regimen consists of approximately a one year treatment with the combination of R-flurbiprofen and SSRI. High and low study doses of a compound of R-flurbiprofen are used (i.e., 800 mg and 400 mg.) A study dose of 800 mg consists of two 400 mg of a R-flurbiprofen as tablets, while a study dose of 400 mg consists one 400 mg placebo and one 400 mg dos of R-flurbiprofen (one therapeutic capsule (or tablet) and one placebo capsule (or tablet)). R-flurbiprofen can be pre-packed into a day-by-day plastic medication dispenser. Treatment with SSRI can be on a one or twice daily dosing regimen, with the dosages described herein.

During the follow-up visit, twelve or fourteen days after beginning treatment, vital signs and adverse side effects of the combination of R-flurbiprofen and SSRI are assessed. In addition, lumbar punctures are performed and blood samples are drawn for laboratory tests and for plasma preparations.

Visits during which lumbar punctures are performed and blood samples are drawn are scheduled for mornings with overnight fasting to avoid obtaining post-prandial or hyperlipemic plasma samples, which can influence levels of Aβ₄₀ and Aβ₄₂. The following paragraph summarizes the biological markers that are analyzed from plasma and CSF samples.

Plasma and CSF biological markers Volume Assay Method Volume of CSF of Plasma Protein, glucose, 1 mL cells Aβ₄₀ ELISA 100 μL×2 100 μL×2 (in duplicate) Aβ₄₂ ELISA 100 μL×2 100 μL×2 (in duplicate) Aβ₃₈. Mass Spectrometry 1 mL Isoprostanes Gas Chromatography/2 mL Mass Spectrometry M-CSF ELISA 50 μL×2 (in duplicate) MCP-1 ELISA 50 μL×2 (in duplicate) Tau, ELISA 50 μL×2 P-tau181 (in duplicate) 50 μL×2 (in duplicate) Plasma levels of the therapeutics compounds of by HPLC 1 mL. The assessment of these markers is within the skill of an ordinary artisan.

Patients having mild-to-moderate Alzheimer's disease undergoing the treatment regimen of this example with R-flurbiprofen in doses of about 10 mg to 1600 mg per day, and a SSRI, can experience a lessening in decline of cognitive function (as measured by ADAS-cog or CDR sum of boxes), plaque pathology, and/or biochemical disease marker progression.

### Example 3

### Treatment of Alzheimer's Disease with R-Flurbiprofen-SSRI Combination

R-flurbiprofen can be administered twice daily as tablets containing 400 mg of active ingredient or as a capsule containing 400 mg of the active ingredient. A higher dose can be administered to the patient in need of such treatment which can involve the patient taking e.g., a 800 mg dose of R-flurbiprofen in the morning and a 800 mg dose of R-flurbiprofen in the evening. Typically, for the treatment of mild-to-moderate Alzheimer's disease, an individual is diagnosed by a doctor as having the disease using a suitable combination of observations. One criterion indicating a likelihood of mild-to-moderate Alzheimer's disease is a score of about 15 to about 26 on the MMSE test. Another criteria indicating mild-to-moderate Alzheimer's disease is a decline in cognitive function. R-flurbiprofen can also be administered in liquid or dosage forms. The dosages can also be divided or modified, and taken with or without food. For example, the 400 mg dose can be divided into two 200 mg tablets or capsules.

Depending on the stage of the disease, the NSAID (i.e., R-flurbiprofen) can also be administered twice daily in liquid, capsule, or tablet dosage forms where the dose has various amounts of R-flurbiprofen (i.e., 850 mg, 750 mg, 700 mg, 650 mg, 600 mg, 550 mg, 500 mg, 450 mg, 350 mg, 300 mg, 250 mg, 200 mg, 150 mg, and 100 mg). Again, the dosages can also be divided or modified, and taken with or without food. The doses can be taken during treatment with other medications for treating Alzheimer's disease or symptoms thereof. For example, the NSAID can be administered twice daily as a tablet containing 400 mg of active ingredient (i.e., R-flurbiprofen) and a SSRI is administered once daily (i.e, a tablet having from about 1 mg to 100 mg fluoxetine; from about 1 mg to about 300 mg of fluvoxamine; from about 1 mg to 100 mg of paroxetine; from about 1 mg to about 200 mg of sertraline; from about 1 mg to about 100 mg citalopram; from about 1 mg to about 50 mg escitalopram oxalate).

Patients having mild-to-moderate Alzheimer's disease undergoing the treatment regimen of this example with R-flurbiprofen doses of about 400 mg to 800 mg and a SSRI can experience a lessening in decline of cognitive function (as measured by the ADAS-cog or CDR sum of boxes), plaque pathology, and/or biochemical disease marker progression.

### Example 4

### Prevention of Alzheimer's Disease with R-Flurbiprofen-SSRI Combination

Prior to the onset of symptoms of Alzheimer's disease or just at the very beginning stages of the disease, patients desiring prophylaxis against Alzheimer's disease can be treated with a combination of R-flurbiprofen and a SSRI. Those needing prophylaxis can be assessed by monitoring assayable disease markers, detection of genes conferring a predisposition to the disease, other risks factors such as age, diet, other disease conditions associated with Alzheimer's disease.

The patient desiring prophylaxis against Alzheimer's disease or prophylaxis of a worsening of the symptoms of Alzheimer's disease can be treated with R-flurbiprofen and a SSRI in an amount sufficient to delay the onset or progression of symptoms of Alzheimer's disease. For example, a patient can be treated with 800 mg of NSAID (i.e., R-flurbiprofen) twice daily and once daily with a SSRI (i.e, a tablet having from about 1 mg to 100 mg fluoxetine; from about 1 mg to about 300 mg of fluvoxamine; from about 1 mg to 100 mg of paroxetine; from about 1 mg to about 200 mg of sertraline; from about 1 mg to about 100 mg citalopram; from about 1 mg to about 50 mg escitalopram oxalate. Alternatively, the SSRI and R-NSAID can be formulated in a single tablet for administration once or twice daily. Another preventive regimen involves administering to the patient 400 mg of R-flurbiprofen twice daily and twice daily with a SSRI (i.e, a total daily dosage of from about 1 mg to 100 mg fluoxetine; from about 1 mg to about 300 mg of fluvoxamine; from about 1 mg to 100 mg of paroxetine; from about 1 mg to about 200 mg of sertraline; from about 1 mg to about 100 mg citalopram; from about 1 mg to about 50 mg escitalopram oxalate The amounts of these active ingredients can be modified to lessen side-effects and/or produce the most therapeutic benefit. For example, 200 mg of R-flurbiprofen twice daily can be administered to reduce sides-effects associated with the use of higher levels of the active ingredient. The preventive treatment can also be, e.g., treatment on alternating days with R-flurbiprofen, or alternating weeks. Lastly, the R-NSAID SSRI combination described herein can be administered to the patient desiring (or needing) prophylaxis against Alzheimer's disease.

### Example 5

### Detection of Amyloid Beta with Biosource Elisa Kit (Camarillo, Calif.)

The present invention provides combination compositions and methods for lowering Aβ₄₂ levels. To test whether the combinations are capable of modulating Aβ levels, a sandwich enzyme-linked immunosorbent assay (ELISA) is employed to measure secreted Aβ (Aβ₄₂ and/or Aβ₄₀) levels. In this example, H4 cells expressing wide type APP695 are seeded at 200,000 cells/ per well in 6 well plates, and incubated at 37° C. with 5% CO₂ overnight. Cells are treated with 1.5 ml medium containing vehicle (DMSO) or a test compounds at 1.25 μM, 2.5 μM, 5.0 μM and 10.0 μM (as well as other concentration if desirable) concentration for 24 hours or 48 hours. The supernatant from treated cells is collected into eppendorf tubes and frozen at −80° C. for future analysis.

The amyloid peptide standard is reconstituted and frozen samples are thawed. The samples and standards are diluted with appropriate diluents and the plate is washed 4 times with Working Wash Buffer and patted dry on a paper towel. 100 μL per well of peptide standards, controls, and dilutions of samples to be analyzed is added. The plate is incubated for 2 hours while shaking on an orbital plate shaker at RT. The plate is then washed 4 times with Working Wash Buffer and patted dry on a paper towel. Detection Antibody Solution is poured into a reservoir and 100 μL /well of Detection Antibody Solution is immediately added to the plate. The plate is incubated at RT for 2 hours while shaking and then washed four times with Working Wash Buffer and patted dry on a paper towel. Secondary Antibody Solution is then poured into a reservoir and 100 μL/well of Secondary Antibody Solution is immediately added to the plate. The plate is incubated at RT for 2 hours with shaking, washed 5 times with Working Wash Buffer, and patted dry on a paper towel.

100 μL of stabilized chromogen is added to each well and the liquid in the wells begins to turn blue. The plate is incubated for 30 minutes at room temperature and in the dark. 100 μL of stop solution is added to each well and the plate is tapped gently to mix resulting in a change of solution color from blue to yellow. The absorbance of each well is read at 450 nm having blanked the plate reader against a chromogen blank composed of 100 μL each of stabilized chromogen and stop solution. The plate is read within 2 hours of adding the stop solution. The absorbance of the standards is plotted against the standard concentration and the concentrations of unknown samples and controls are calculated.

Plasma and CSF samples obtained from patients can also be analyzed for Aβ₄₂, Aβ₃₈ or Aβ₄₀ and other Aβ peptide levels in a similar manner.

### Example 6

### Detection of Amyloid Beta with Innogenetic Elisa Kit (Gent, Belgium)

The present invention provides combination compositions and methods for lowering Aβ₄₂ levels. To test whether the combination methods and compositions are capable of modulating Aβ levels, sandwich enzyme-linked immunosorbent assay (ELISA) is employed to measure secreted Aβ (Aβ₄₂ and/or Aβ₄₀) levels. In this example, H4 cells expressing wide type APP695 are seeded at 200,000 cells/ per well in 6 well plates, and incubated at 37° C. with 5% CO₂ overnight. Cells are treated with 1.5 ml medium containing vehicle (DMSO) or test compounds at 1.25 μm, 2.5 μm, 5.0 μm and 10.0 μm concentration for 24 hours or 48 hours. The supernatant from treated cells is collected into eppendorf tubes and frozen at −80 ° C. for future analysis.

130 μl per well of samples, standards, and blanks is added to a 96-well polypropylene plate. 200 μl of samples, standards, and blanks from the polypropylene plate is added to the antibody-coated plates. The strip-holder with the appropriate number of strips is applied to the antibody-coated plates and the strips are covered with an adhesive sealer. The plate is then incubated 3 hours at room temperature while shaking on an orbital plate shaker.

The first antibody solution is prepared with Conjugate Diluent 1 at 1:100 ratio. Each well of the antibody-coated plates is washed 5 times with 400 μl washing solution and 100 μl of the prepared first antibody solution is added to each well. The strips are applied to the plate, covered with an adhesive sealer, and the plate is incubated for 1 hour at room temperature while shaking on an orbital plate shaker.

The second antibody (conjugate 2) solution is prepared with Conjugate Diluent 2 at 1:100 ratio. Each well of the antibody-coated plates are washed 5 times with 400 μl washing solution and 100 μl of the prepared second antibody solution is added to each well. The strips are applied, covered with an adhesive sealer, and the plate is incubated 30 min at room temperature while shaking on an orbital plate shaker. Each well of the antibody-coated plates is then are washed for 5 times with 400 μl washing solution.

A substrate solution is prepared by diluting Substrate 100× with HRP Substrate Buffer. 100 μl of the prepared substrate solution is added to each well of the antibody-coated plate. The strips are applied, covered with an adhesive sealer, and the plate is incubated for 30 min at room temperature. 100 μl Stop Solution is then added to each well to stop the reaction. The strip-holder is carefully taped to ensure through mixing. The reader is blanked and the absorbance of the solution in the wells is read at 450 nm. The absorbance of the standards is plotted against the standard concentration and the concentration of samples is calculated using the standard curve.

Plasma and CSF samples obtained from patients can also be analyzed for Aβ₄₂, Aβ₃₈ or Aβ₄₀ and other Aβ peptide levels in a similar manner.

### Example 7

### Neuroprotection Assay

The present invention provides combination compositions and methods for slowing the death or decline of neurons. To test the ability of combination compositions and methods of the present invention to protect against neurotoxicity, adult female Sprague Dawley rats are obtained and injected intraperitoneally with various doses of a combination therapeutics of the present invention. At the same time, the test animals also receive a subcutaneous injection of MK-801 (0.5 mg/kg), which has been shown to consistently induce, in all treated rats, a fully developed neurotoxic reaction consisting of acute vacuole formation in the majority of pyramidal neurons in layers III and IV of the posterior cingulate and retrosplenial (PC/RS) cortices.

Control animals are administered the liquid which was used to dissolve the test agent and the same dosage of MK-801 (0.5 mg/kg sc). The animals are sacrificed four hours after treatment and the number of vacuolated PC/RS neurons are counted on each side of the brain, at a rostrocaudal level immediately posterior to where the corpus callosum ceases decussating across the midline (approximately 5.6 mm caudal to bregma). The toxic reaction approaches maximal severity at this level and shows very little variability between different animals.

Percentage reduction in neurotoxicity is calculated by dividing the mean number of vacuolated neurons in a given treatment group, by the mean number of vacuolated neurons in control animals that were treated with MK-801 but not the protective agent. The result is subtracted from one and multiplied by 100, to calculate a percentage. Linear regression analysis can be used to determine an ED₅₀ (i.e., the dosage of a given compound that reduces the mean number of vacuolated neurons to 50% of the value in control animals), with the 25th and 75th percentiles defining the confidence limits.

### Example 8

### Combination Treatment of Animals: Determine the Combination's Effect on Levels of Aβ₄₂ and Alzheimer's Disease Progression

To determine the effect of a combination therapy of the present invention on levels of Aβ₄₂ and Alzheimer's disease, an animal is treated with the combination of therapeutics and the levels of Aβ₄₂ in the brain are measured. Three month-old TG2576 mice that overexpress APP(695) with the “Swedish” mutation (APP695NL) are used. Mice overexpressing APP(695) with the “Swedish” mutation have high levels of soluble Aβ in the their brains and develop memory deficits and plaques with age, making them suitable for examining the effect of compounds on levels of Aβ₄₂ and Alzheimer's Disease. “Test” TG25276 mice are treated with the therapeutic combination and “control” TG25276 mice are not. The brain levels of SDS-soluble Aβ₄₀ and Aβ₄₂ for “test” mice are compared to “control” mice using ELISA. Test mice that have a reduction in Aβ₄₂ levels suggest that treatment with the combination prevents or slows amyloid pathology by decreasing the ratio of Aβ₄₂ to Aβ₄₀ in the brain.

### Example 9

### Combination Treatment of Animals: Determine the Combination's Effect on Memory and Alzheimer′s Disease.

The present invention provides combination compositions and methods for treating or preventing Alzheimer's disease. To test the effect of compositions of the present invention on memory and Alzheimer's disease, TG2576 mice that overexpress APP(695) with the “Swedish” mutation (APP695NL) are used. Mice overexpressing APP(695) with the “Swedish” mutation develop memory deficits and plaques with age, making them suitable for examining the effect of compounds on memory and Alzheimer's Disease. The test compounds are administered daily for two weeks to test groups of the TG2576 mice in age groups of: 1) 4-5 months, 2) 6-11 months, 3) 12-18 months, and 4) 20-25 months. Groups of control TG2576 mice of corresponding ages are not administered the compound. Both control and test groups then have memory tested in a version of the Morris water maze (Norris, J. Neurosci. Methods, 11:47-60 (1984)) that is modified for mice. The water maze contains a metal circular pool of about 40 cm in height and 75 cm in diameter. The walls of the pool have fixed spatial orientation clues of distinct patterns or shelves containing objects. The pool is filled with room temperature water to a depth of 25 cm and an escape platform is hidden 0.5 cm below the surface of the 25-cm-deep water at a fixed position in the center of one of the southwest quadrant of pool. The test and control mice are trained for 10 days in daily sessions consisting of four trials in which the mouse starts in a different quadrant of the pool for each trial. The mice are timed and given 60 seconds to find the escape platform in the pool. If the mice have not found the escape platform after 60 seconds, they are guided into it. The mice are then allowed to rest on the platform for 30 seconds and the amount of time it takes the mice to find the platform is recorded. Probe trials are run at the end of the trials on the 4th, 7th, and 10th days of training, in which the platform is removed and the mice are allowed to search for the platform for 60 sec. The percentage of time spent in the quadrant where the platform was in previous trials is calculated.

In training trials, the time it takes test group mice to reach the escape platform is compared to the time taken by control group mice of corresponding ages. In probe trials, the percentage of time spent by test group mice in the quadrant where the platform was in previous trials is compared to the percentage time spent by control mice. Quicker location of the escape platform in training trials and/or an increased percentage time spent in the previous quadrant of the maze during probe trials is indicative of spatial learning and memory. Because memory loss is a hallmark of Alzheimer's disease, test mice that have better learning and memory when compared to control mice indicate that the combination can be effective in treating or slowing Alzheimer's disease and/or its symptoms.

### Example 10

### Determination of COX Inhibition Activity

In vitro cellular COX inhibition can be determined using specific assays for inhibition of COX-1 and COX-2 (Kalgutkar et al. J. Med Chem, 43:2860-2870 (2000)). Another art-known cellular assay for determining COX inhibition is based on the production of prostaglandin-E₂ from exogenous arachidonic acid in cells expressing COX-1, COX-2, or a combination thereof. COX enzymes (prostaglandin H synthase) catalyze the rate-limiting step in prostaglandin synthesis from arachidonic acid. Cell lines are known and available that express at least one form of the enzyme. For example, a human skin fibroblast line can be induced with IL-1 to synthesize COX-2, and a kidney epithelial cell line 293 has been stably transfected to constitutively express COX-1. In these assays, arachidonic acid can be added exogenously to increase signal to readably detectable levels. Thus, the amount of prostaglandin-E₂ in the extracellular medium can be assayed by radioimmunoassay, for measuring COX activity. IC₅₀ values for compounds for COX-1 and COX-2 can be determined by an ordinary skilled artisan. Anti-inflammatory activities of compounds can be determined using the art-known rat/mouse paw edema assay as described in Penning et al. J. Med Chem., 40:1347-1365 (1997).

For a further description of assays, cell line, and techniques capable of assessing COX inhibitory activity and Aβ₄₂ lowering activity see, e.g., WO 01/78721, and references cited therein, all of which are incorporated herein in their entirety.

What is claimed is:

1. A pharmaceutical composition comprising a combination of R-flurbiprofen or a pharmaceutically acceptable salt or ester thereof and an SSRI or a pharmaceutically acceptable salt thereof.

2. The composition of claim 1, comprising about 400 mg R-flurbiprofen or an equivalent amount of a pharmaceutically acceptable salt thereof.

3. The composition of claim 2, wherein said composition is substantially free of S-flurbiprofen.

4. The composition of claim 1, comprising about 800 mg R-flurbiprofen or an equivalent amount of a pharmaceutically acceptable salt thereof.

5. The composition of claim 4, wherein said composition is substantially free of S-flurbiprofen.

6. The composition of claim 1, wherein said SSRI is a sertraline.

7. The composition of claim 1, wherein said SSRI is selected from the group consisting of fluoxetine, fluvoxamine, paroxetine, sertraline, citalopram, and escitalopram, and pharmaceutically acceptable salts thereof.

8. The composition of claim 1, comprising an amount of said R-flurbiprofen or pharmaceutically acceptable salt or ester thereof when administered in a single dose to a fasting individual sufficient to produce a plasma Cₘₐₓ of about 25-150 μg per mL per dose and an AUC (area under curve of concentration versus time; total drug exposure) of from about 200 hr·μg/mL to about 600 hr·μg/mL.

9. A method of treating or delaying the onset of dementia such as Alzheimer's disease in an individual comprising treating the individual with R-flurbiprofen or a pharmaceutically acceptable salt or ester thereof and an SSRI or a pharmaceutically acceptable salt thereof, at an effective amount sufficient to treat or delay the onset of Alzheimer's disease.

10. The method of claim 9, wherein said individual is diagnosed of mild cognitive impairment.

11. The method of claim 9, wherein said individual is diagnosed of mild to moderate Alzheimer's disease.

12. The method of claim 9, wherein said individual is also diagnosed of depression.

13. The method of claim 9, wherein said SSRI is selected from the group consisting of fluoxetine, fluvoxamine, paroxetine, sertraline, citalopram, sertraline and escitalopram, and pharmaceutically acceptable salts thereof.

14. The method of claim 9, wherein said composition comprises about 400 mg R-flurbiprofen or an equivalent amount of a pharmaceutically acceptable salt thereof.

15. The method of claim 14, wherein said composition is substantially free of S-flurbiprofen.

16. The method of claim 9, wherein said composition comprises about 800 mg R-flurbiprofen or an equivalent amount of a pharmaceutically acceptable salt thereof.

17. The method of claim 16, wherein said composition is substantially free of S-flurbiprofen.

18. The method of claim 9, wherein said individual is treated such that a plasma R-flurbiprofen Cₘₐₓ of about 25-150 μg per mL per dose and a plasma R-flurbiprofen AUC₁₂ (area under curve of concentration in a 12-hour window, i.e., total drug exposure in a 12-hour window) of from about 200 hr·μg/mL to about 450 hr·μg/mL are achieved.
